(19) United States
(12) Patent Application Publication
(10) Pub. No.: US 2022/0387417 A1
(43) Pub. Date: Dec. 8, 2022

(54) Pharmaceutical Combination and Use Thereof

(71) Applicants: CSTONE PHARMACEUTICALS (SUZHOU) CO., LTD., Jiangsu (CN); CSTONE PHARMACEUTICALS (SHANGHAI) CO., LTD., Shanghai (CN); CSTONE PHARMACEUTICALS, Grand Cayman (GB)

(72) Inventor: Juan Zhang, Jiangsu (CN)

(21) Appl. No.: 17/776,179

(22) PCT Filed: Nov. 11, 2020
(86) PCT No.: PCT/CN2020/128083

§ 371 Date: May 11, 2022

(30) Foreign Application Priority Data
Nov. 11, 2019 (CN) PCT/CN2019/117155

(51) Int. Cl. A61K 31/47; A61K 39/00; A61K 9/00; A61P 35/00

(52) U.S. Cl. CPC A61K 31/47; A61K 39/001111; A61K 9/0019; A61P 35/00

(57) ABSTRACT

The present disclosure relates to a novel pharmaceutical combination and a use thereof. The pharmaceutical combination can be used to treat cancer, such as colon cancer.

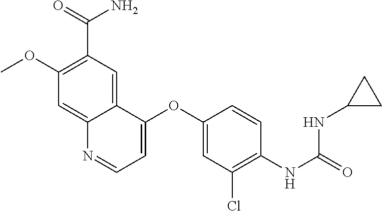 [SMILES: COc1cc2nccc(Oc3ccc(NC(=O)NC4CC4)c(Cl)c3)c2cc1C(N)=O]

### CROSS REFERENCE TO RELATED APPLICATION

The present application claims the priority of PCT/CN2019/117155, filed on Nov. 11, 2019, the contents of which are incorporated herein by reference in their entireties.

### FIELD OF THE INVENTION

The present disclosure relates to a pharmaceutical combination and use thereof.

### BACKGROUND OF THE INVENTION

Colon cancer has one of the highest mortality rates of any malignant disease globally. According to the annual, age-adjusted cancer incidence and mortality rate in the USA from 1975 to 2002, colon cancer was among the three most frequently diagnosed types of cancer. There is a continuing need for treating colon cancer.

Lenvatinib (CAS Number 417716-92-8) is an active inhibitor of multiple receptor tyrosine kinases (e.g., receptor tyrosine kinases involved in angiogenesis and tumor proliferation) including vascular endothelial growth factor (VEGF), fibroblast growth factor (FGF), platelet derived growth factor receptorα (PDGFRα), KIT, and RET proto-oncogene receptors. It has been approved by US FDA for the treatment of thyroid cancer on 2005, in the form of its mesylate salt (CAS Number 857890-39-2).

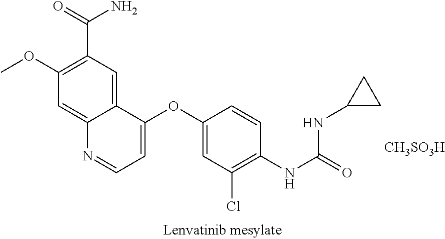

### CONTENT OF THE INVENTION

The present disclosure relates to a novel pharmaceutical combination and use thereof.

In one aspect, the present disclosure provides a pharmaceutical combination comprising a substance A and a substance B;

wherein the substance A is Compound F, a crystal form thereof, a pharmaceutically acceptable salt thereof or a solvate thereof;

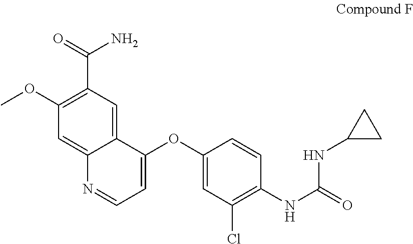 [SMILES: COc1cc2nccc(Oc3ccc(NC(=O)NC4CC4)c(Cl)c3)c2cc1C(N)=O]

the substance B is an antibody M comprising a heavy chain CDR amino acid sequence selected from the group consisting of: SEQ ID NOs: 1, 3, 5, 7, 9, 11, 13, 15, 17, 19, 21 and 23, or an antigen binding fragment thereof.

The pharmaceutical combination as defined above can further comprise a pharmaceutical excipient.

The pharmaceutical combination as defined above can be in the form of a fixed combination or a non-fixed combination. In some embodiments, the pharmaceutical combination is in the form of a non-fixed combination.

In some embodiments, the antibody M may comprise:

(a) a heavy chain variable region comprising a HCDR1 amino acid sequence of SEQ ID NO: 1, a HCDR2 amino acid sequence of SEQ ID NO: 3, and a HCDR3 amino acid sequence of SEQ ID NO: 5; and a light chain variable region comprising a LCDR1 amino acid sequence of SEQ ID NO: 2, a LCDR2 amino acid sequence of SEQ ID NO: 4, and a LCDR3 amino acid sequence of SEQ ID NO: 6;

(b) a heavy chain variable region comprising a HCDR1 amino acid sequence of SEQ ID NO: 7, a HCDR2 amino acid sequence of SEQ ID NO: 9, and a HCDR3 amino acid sequence of SEQ ID NO: 11; and a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12;

(c) a heavy chain variable region comprising a HCDR1 amino acid sequence of SEQ ID NO: 13, a HCDR2 amino acid sequence of SEQ ID NO: 15, and a HCDR3 amino acid sequence of SEQ ID NO: 17; and a light chain variable region comprising a LCDR1 amino acid sequence of SEQ ID NO: 14, a LCDR2 amino acid sequence of SEQ ID NO: 16, and a LCDR3 amino acid sequence of SEQ ID NO: 18; or

(d) a heavy chain variable region comprising a HCDR1 amino acid sequence of SEQ ID NO: 19, a HCDR2 amino acid sequence of SEQ ID NO: 21, and a HCDR3 amino acid sequence of SEQ ID NO: 23; and a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12.

In some embodiments, the antibody M may comprise:

(a) a heavy chain variable region comprising a HCDR1 amino acid sequence of SEQ ID NO: 7, a HCDR2 amino acid sequence of SEQ ID NO: 9, and a HCDR3 amino acid sequence of SEQ ID NO: 11; and

(b) a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12.

In some embodiments, the antibody M may comprise:

(a) a heavy chain variable region comprising an amino acid sequence of SEQ ID NO: 20; and a light chain variable region comprising an amino acid sequence of SEQ ID NO: 22;

(b) a heavy chain variable region comprising an amino acid sequence of SEQ ID NO: 24; and a light chain variable region comprising an amino acid sequence of SEQ ID NO: 25;

(c) a heavy chain variable region comprising an amino acid sequence of SEQ ID NO: 26; and a light chain variable region comprising an amino acid sequence of SEQ ID NO: 27; or

(d) a heavy chain variable region comprising an amino acid sequence of SEQ ID NO: 28; and a light chain variable region comprising an amino acid sequence of SEQ ID NO: 25.

In some embodiments, the antibody M may comprise:

(a) a heavy chain variable region comprising an amino acid sequence of SEQ ID NO: 24; and

(b) a light chain variable region comprising an amino acid sequence of SEQ ID NO: 25.

CDR amino acid sequences of exemplary antibody M, fully human monoclonal antibodies 1.4.1, 1.14.4, 1.20.15, and 1.46.11 are shown in Table 1.

TABLE 1

| | CDR1 | CDR2 | CDR3 |
|---|---|---|---|
| 1.4. | SEQ ID NO: 1 | SEQ ID NO: 3 | SEQ ID NO: 5 |
| 1-VH | IRTYYWG | YIYYSGSTRYNPSLKS | LSYFFDY |
| 1.4. | SEQ ID NO: 2 | SEQ ID NO: 4 | SEQ ID NO: 6 |
| 1-VL | SGDKLGDKYAC | QDTKRPS | QAWDSGTVI |
| 1.14. | SEQ ID NO: 7 | SEQ ID NO: 9 | SEQ ID NO: 11 |
| 4-VH | SYAMS | GISGSGGFTYYADSVKG | PPRGYNYGPFDY |
| 1.14. | SEQ ID NO: 8 | SEQ ID NO: 10 | SEQ ID NO: 12 |
| 4-VL | GGNNIGSKSVH | DDSDRPS | QVWDSSSDHVV |
| 1.20. | SEQ ID NO: 13 | SEQ ID NO: 15 | SEQ ID NO: 17 |
| 15-VH | SISNYWG | SIYYSGSTNYNPPLKS | LTYYFDY |
| 1.20. | SEQ ID NO: 14 | SEQ ID NO: 16 | SEQ ID NO: 18 |
| 15-VL | SGDKLGDKYAC | QDSKRPS | QTWDSSTVV |
| 1.46. | SEQ ID NO: 19 | SEQ ID NO: 21 | SEQ ID NO: 23 |
| 11-VH | SYAMS | GFSGSGFITYYADSVKG | PPRGYNYGPFDY |
| 1.46. | SEQ ID NO: 8 | SEQ ID NO: 10 | SEQ ID NO: 12 |
| 11-VL | GGNNIGSKSVH | DDSDRPS | QVWDSSSDHVV |

Exemplary antibody M, fully human monoclonal antibodies 1.4.1, 1.14.4, 1.20.15, and 1.46.11, have heavy chain variable region and light chain variable region as shown in the following Table 2, and a constant region of human IgG4 isotype.

TABLE 2

| | | SEQ | |
|---|---|---|---|
| | | ID | |
| Clone ID | | NO | Amino acid sequence |
| 1.4.1 | VH | 20 | QLQLQESGPGLVKPSESLSLTCTVSGGSISIRTYY |
| | | | WGWIRQPPGTGLEWMGYIYYSGSTRYNPSLKS |
| | | | RVTISVDTSKNQFSLKLSSVTAADTAVYYCARLS |
| | | | YFFDYWGQGTLVTVSS |
| | VL | 22 | SYELTQPPSVSVSPGQTASITCSGDKLGDKYAC |
| | | | WYQQKPGQSPVMVIYQDTKRPSGIPERFSGSNS |
| | | | GNTATLTISGTLAMDEADYYCQAWDSGTVIFGG |
| | | | GTKLTVL |
| 1.14.4 | VH | 24 | EVQLLESGGGLVQPGGSLRLSCAASGFTFSSYA |
| | | | MSWVRQAPGKGLEWVSGISGSGGFTYYADSVK |
| | | | GRFTISRDNSKNTLYLQMNSLRAEDTAVYYCAK |
| | | | PPRGYNYGPFDYWGQGTLVTVSS |
| | VL | 25 | SYVLTQPPSVSVAPGQTARITCGGNNIGSKSVH |
| | | | WYQQKPGQAPVLVVYDDSDRPSGIPERFSGSNS |
| | | | GNTATLTISRVEAGDEADYYCQVWDSSSDHVVF |
| | | | GGGTKLTVL |
| 1.20.15 | VH | 26 | QLQLQESGPGLVKPSETLSLTCTVSGGSISSISNY |
| | | | WGWIRQPPGKGLEWIGSIYYSGSTNYNPPLKSR |
| | | | VTISVDTTKNQFSLKLSSVTAADTAVYYCARLT |
| | | | YYFDYWGQGMLVTVSS |
| | VL | 27 | SYDLTQPPSVSVSPGQTASITCSGDKLGDKYAC |
| | | | WYQQKPGQSPLLVIQQDSKRPSGIPARFSGSNSG |
| | | | NTATLTISGTQAMDEADYFCQTWDSSTVVFGG |
| | | | GTKLTVL |
| 1.46.11 | VH | 28 | EVQLLESGGGLVQPGGSLRLSCAASGFTFSSYA |
| | | | MSWVRQAPGKGLEWVSGFSGSGFITYYADSVK |
| | | | GRFTISRDNSKNTLYLQMNSLRAEDTAVYYCA |
| | | | MPPRGYNYGPFDYWGQGTLVTVSS |
| | VL | 25 | SYVLTQPPSVSVAPGQTARITCGGNNIGSKSVH |
| | | | WYQQKPGQAPVLVVYDDSDRPSGIPERFSGSNS |
| | | | GNTATLTISRVEAGDEADYYCQVWDSSSDHVVF |
| | | | GGGTKLTVL |

In some embodiments, the antibody M may be a camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a ds diabody, a nanobody, a domain antibody, or a bivalent domain antibody.

In some embodiments, the antibody M may be a monoclonal antibody, fully human antibody, humanized antibody, chimeric antibody, recombinant antibody, bispecific antibody, labeled antibody, bivalent antibody, or anti-idiotypic antibody.

In some embodiments, the antibody M may be fully human antibody. In some embodiments, the fully human antibody is prepared using recombinant methods. For example, transgenic animal such as a mouse can be made to carry transgenes or transchromosomes of human immunoglobulin genes, and therefore capable of producing fully human antibodies after immunization with proper antigen such as human PD-L1. Fully human antibodies can be isolated from such transgenic animal, or alternatively, can be made by hybridoma technology by fusing the spleen cells of the transgenic animal with an immortal cell line to generate hybridoma cells secreting the fully human antibodies. Exemplary transgenic animals include, without limitation, OmniRat, whose endogenous expression of rat immunoglobulin genes are inactivated and at the same time engineered to contain functional recombinant human immunoglobulin loci; OmniMouse, whose endogenous expression of mouse immunoglobulin genes are inactivated and at the same time engineered to contain recombinant human immunoglobulin loci having J-locus deletion and a C-kappa mutation; OmniFlic, which is a transgenic rat whose endogenous expression of rat immunoglobulin genes are inactivated and at the same time engineered to contain recombinant human immunoglobulin loci having a single common, rearranged VkJk light chain and functional heavy chain. Detailed information can be further found at: Osborn M. et al, Journal of Immunology, 2013, 190: 1481-90; Ma B. et al, Journal of Immunological Methods 400-401 (2013) 78-86; Geurts A. et al, Science, 2009, 325:433; U.S. Pat. No. 8,907,157; EP patent 2152880B1; EP patent 2336329B1, all of which are incorporated herein by reference to its entirety. Other suitable transgenic animals can also be used, for example, HuMab mice (see, for details, Lonberg, N. et al. Nature 368(6474): 856 859 (1994)), Xeno-Mouse (Mendez et al. Nat Genet., 1997, 15:146-156), TransChromo Mouse (Ishida et al. Cloning Stem Cells, 2002, 4:91-102) and VelocImmune Mouse (Murphy et al. Proc Natl Acad Sci USA, 2014, 111:5153-5158), Kymouse (Lee et al. Nat Biotechnol, 2014, 32:356-363), and transgenic rabbit (Flisikowska et al. PLoS One, 2011, 6:e21045). In some embodiments, the antibody M is fully human monoclonal antibody.

In some embodiments, the antibody M may further comprise an immunoglobulin (e.g., human IgG4) constant region. In some embodiments, the antibody M may further comprise a heavy chain constant region of human IgG4 (e.g., IgG4 isotype), and a light chain constant region of human λ or κ light chain. In some embodiments, the constant region may further comprise one or more modifications to confer desirable properties. For example, the constant region may be modified to reduce or deplete one or more effector functions, to improve FcRn receptor binding, or to introduce one or more cysteine residues.

In some embodiments, the antibody M may further comprise a conjugate. It is contemplated that a variety of conjugates may be linked to the antibodies or antigen-binding fragments (see, for example, “Conjugate Vaccines”, Contributions to Microbiology and Immunology, J. M. Cruse and R. E. Lewis, Jr. (eds.), Carger Press, New York, (1989)). These conjugates may be linked to the antibodies or antigen-binding fragments by covalent binding, affinity binding, intercalation, coordinate binding, complexation, association, blending, or addition, among other methods. In some embodiments, the antibody M may be engineered to contain specific sites outside the epitope binding portion that may be utilized for binding to one or more conjugates. For example, such a site may include one or more reactive amino acid residues, such as for example cysteine or histidine residues, to facilitate covalent linkage to a conjugate. In some embodiments, the antibodies may be linked to a conjugate indirectly, or through another conjugate. For example, the antibody or an antigen-binding fragment thereof may be conjugated to biotin, then indirectly conjugated to a second conjugate that is conjugated to avidin. The conjugate can be a detectable label, a pharmacokinetic modifying moiety, a purification moiety, or a cytotoxic moiety. Examples of detectable label may include a fluorescent labels (e.g. fluorescein, rhodamine, dansyl, phycoerythrin, or Texas Red), enzyme-substrate labels (e.g. horseradish peroxidase, alkaline phosphatase, luceriferases, glucoamylase, lysozyme, saccharide oxidases or β-D-galactosidase), radioisotopes (e.g. ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides, luminescent labels), chromophoric moiety, digoxigenin, biotin/avidin, a DNA molecule or gold for detection. In certain embodiments, the conjugate can be a pharmacokinetic modifying moiety such as PEG which helps increase half-life of the antibody. Other suitable polymers include, such as, carboxymethylcellulose, dextran, polyvinyl alcohol, polyvinyl pyrrolidone, copolymers of ethylene glycol/propylene glycol, and the like. In some embodiments, the conjugate can be a purification moiety such as a magnetic bead. A “cytotoxic moiety” can be any agent that is detrimental to cells or that can damage or kill cells. Examples of cytotoxic moiety include, without limitation, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

In some embodiments, the antibody M may be anti-PD-L1 antibody.

In some embodiments, the antibody M may be capable of specifically binding to human PD-L1. In some embodiments, the antibody M is capable of specifically binding to human PD-L1 with a binding affinity (Kd) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, about 10⁻¹⁰ M, 10⁻¹⁰ M to 10^{-8.5} M, or 10⁻¹⁰ M to 10⁻⁸ M) as measured by plasmon resonance binding assay. The binding affinity can be represented by K_{D} value, which is calculated as the ratio of dissociation rate to association rate (k_{off}/kₒₙ) when the binding between the antigen and the antigen-binding molecule reaches equilibrium. The antigen-binding affinity (e.g. K_{D}) can be appropriately determined using suitable methods known in the art, including, for example, plasmon resonance binding assay using instruments such as Biacore (see, for example, Murphy, M. et al, Current protocols in protein science, Chapter 19, unit 19.14, 2006).

In certain embodiments, the antibody M may be capable of specifically binding to human PD-L1 with an EC₅₀ (i.e., 50% binding concentration) of 0.1 nM-100 nM (e.g., 0.1 nM-50 nM, 0.1 nM-30 nM, 0.1 nM-20 nM, 0.1 nM-10 nM, or 0.1 nM-1 nM. Binding of the antibodies to human PD-L1 can be measured by methods known in the art, for example, sandwich assay such as ELISA, Western Blot, FACS or other binding assay. In an illustrative example, the test antibody (i.e., first antibody) is allowed to bind to immobilized human PD-L1 or cells expressing human PD-L1, after washing away the unbound antibody, a labeled secondary antibody is introduced which can bind to and thus allow detection of the bound first antibody. The detection can be conducted with a microplate reader when immobilized PD-L1 is used, or by using FACS analysis when cells expressing human PD-L1 are used. In some embodiments, the anti-PD-L1 is capable of specifically binding to human PD-L1 with an EC₅₀ (i.e. 50% effective concentration) of 1 nM to 10 nM, or 1 nM to 5 nM as measured by FACS analysis.

In some embodiments, the substance A can be the mesylate of Compound F.

In some embodiments, the substance B can be the antibody M.

Pharmaceutical Composition, Kit and Use of the Substance A and the Substance B in Combination

In another aspect, the present disclosure also provides a pharmaceutical composition comprising a substance A and a substance B; wherein the substance A and the substance B are as defined above.

The pharmaceutical composition may further comprise a pharmaceutical excipient.

In another aspect, the present disclosure also provides a kit comprising:

a first container comprising a first pharmaceutical composition comprising a substance A; and

a second container comprising a second pharmaceutical composition comprising a substance B;

wherein the substance A and the substance B are as defined above.

The first pharmaceutical composition may further comprise a pharmaceutical excipient.

The second pharmaceutical composition may further comprise a pharmaceutical excipient.

In another aspect, the present disclosure also provides a use of the pharmaceutical combination or the pharmaceutical composition as defined above in manufacturing a medicament for treating a cancer.

In another aspect, the present disclosure also provides a method for treating a cancer comprising administering (e.g., a therapeutically effective amount of) the pharmaceutical combination or the pharmaceutical composition as defined above (e.g., to a subject (e.g., a human or a mouse) in need thereof).

In some embodiments, the cancer can be non-small cell lung cancer, small cell lung cancer, renal cell cancer, colorectal cancer, colon cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas or myelomas.

In some embodiments, the cancer can be colon cancer.

Dosages Regimens of the Substance A and the Substance B in Combination

The substance A and the substance B can be administrated simultaneously or separately.

In some embodiments, the substance A and the substance B can be administrated separately.

The term “administrated simultaneously” means administration at a same time point. The substance A and the substance B can be present in a single pharmaceutical composition (e.g., in a single dosage form, e.g., in one capsule) to be administrated at a same time point; or, the substance A and the substance B can also be present separately in different pharmaceutical compositions to be administrated at a same time point.

The term “administrated separately” means administration at different time points. For example, the substance A and the substance B can be present separately in different pharmaceutical compositions to be administrated at different time points. The separated administration may be close in time or distant in time but make sure the substance A and the substance B can act in concert so as to provide the desired therapeutic effect. For example, the substance A can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the substance B, to a subject in need thereof. In some embodiments, the substance A and the substance B can be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In some embodiments, the substance A and the substance B can be administered 30 minutes to 1 hour apart.

Whether the substance A and the substance B are administrated simultaneously or separately, the dosage regimens (e.g., route, dose and interval of administration) of the substance A and the substance B can be the same or different, which can be adjusted by a person skilled in the art in order to provide an optimal therapeutic effect as needed.

Suitable routes of administration for the substance A and the substance B includes gastrointestinal administration (e.g., oral administration) and parenteral administration (e.g., injection, e.g., intraperitoneal injection, intravenous injection, subcutaneous injection, or intramuscular injection).

Oral administration may involve swallowing, so that the substance enters the gastrointestinal tract, and/or buccal, lingual, or sublingual administration by which the substance enters the blood stream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches. Further, the substance can be administered as a spray dried dispersion. Solid formulations for oral administration may be formulated to be immediate and/or modified release. Modified release formulations include delayed-, sustained-, pulsed-, controlled-, targeted and programmed release. Liquid formulations include suspensions, solutions, syrups and elixirs. Such formulations may be employed as fillers in soft or hard capsules (made, for example, from gelatin or hydroxypropyl methylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques. Formulations for parenteral administration may be formulated to be immediate and/or modified release. Modified release formulations include delayed-, sustained-, pulsed-, controlled-, targeted and programmed release.

In some embodiments, the substance A, e.g., the mesylate of Compound F, is administrated orally.

In some embodiments, the substance B, e.g., the antibody M, is administrated by injection, e.g., intraperitoneal injection, intravenous injection, subcutaneous injection, or intramuscular injection, e.g., intraperitoneal injection.

The substance A can be administered (e.g., orally) at a dose based on the body weight of a subject, non-limiting examples of the dose can range from 0.01 to 50 mg/kg, e.g., 0.05 mg/kg, 0.1 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 25 mg/kg, 30 mg/kg, 35 mg/kg, 40 mg/kg, 45 mg/kg or 50 mg/kg (e.g., based on the mesylate of Compound F). In some embodiments, the substance A (e.g., the mesylate of Compound F) is administered at a dose of 1-10 mg/kg, e.g., 1 mg/kg. The above dose of the substance A can be administered to a subject in a frequency of QD (once a day), BID (twice a day), TID (three times a day), Q2D (once every two days), Q3D (once every three days), Q4D (once every four days), QW (once a week), BIW (twice a week) or Q2W (once every two weeks). In some embodiments, the above dose of the substance A (e.g., the mesylate of Compound F) is administrated in a frequency of QD.

In some embodiments, the substance A, e.g., the mesylate of Compound F, is administrated (e.g., orally) to a subject (e.g., a human or a mouse) at a dose of 1 mg/kg, QD.

The substance A can also be administered (e.g., orally) to a subject in a fixed (e.g., predetermined) dose. Non-limiting examples of the fixed dose can range from 0.1-1000 mg, e.g., 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mg (e.g., based on the mesylate of Compound F). In some embodiments, the substance A (e.g., the mesylate of Compound F) is administered at a dose of 10-30 mg, e.g., 20 mg. The above fixed dose of the substance A can be administrated to the subject in a frequency of QD, BID, TID, Q2D, Q3D, Q4D, QW, BIW or Q2W. In some embodiments, the above fixed doses of the substance A (e.g., the mesylate of Compound F) is administrated in a frequency of QD.

In some embodiments, the substance A, e.g., the mesylate of Compound F, is administrated (e.g., orally) to a subject (e.g., a human or a mouse) at a dose of 10-30 mg, QD.

The substance B can be administered (e.g., by injection, e.g., intraperitoneal injection, intravenous injection, subcutaneous injection, or intramuscular injection, e.g., intraperitoneal injection) at a dose based on the body weight of a subject, non-limiting examples of the dose can range from 0.01 to 50 mg/kg, e.g., 0.05 mg/kg, 0.1 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 25 mg/kg, 30 mg/kg, 35 mg/kg, 40 mg/kg, 45 mg/kg or 50 mg/kg (e.g., based on the mesylate of Compound F). In some embodiments, the substance B is administered at a dose of 1-10 mg/kg, e.g., 3 mg/kg. The above dose of the substance B can be administered to a subject in a frequency of QD, BID, TID, Q2D, Q3D, Q4D, QW, BIW or Q2W. In some embodiments, the above dose of the substance B is administrated in a frequency of Q2D.

In some embodiments, the substance B is administrated (e.g., by injection, e.g., intraperitoneal injection, intravenous injection, subcutaneous injection, or intramuscular injection, e.g., intraperitoneal injection) to a subject (e.g., a human or a mouse) at a dose of 3 mg/kg, Q2D.

The substance B can also be administered (e.g., by injection, e.g., intraperitoneal injection, intravenous injection, subcutaneous injection, or intramuscular injection, e.g., intraperitoneal injection) to a subject in a fixed (e.g., predetermined) dose. Non-limiting examples of the fixed dose can range from 0.1-1000 mg, e.g., 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mg. In some embodiments, the substance B is administered at a dose of 10-80 mg. The above fixed dose of the substance B can be administrated to the subject in a frequency of QD, BID, TID, Q2D, Q3D, Q4D, QW, BIW or Q2W. In some embodiments, the above fixed doses of the substance B is administrated in a frequency of Q2D.

In some embodiments, the substance B is administrated (e.g., by injection, e.g., intraperitoneal injection, intravenous injection, subcutaneous injection, or intramuscular injection, e.g., intraperitoneal injection) to a subject (e.g., a human or a mouse) at a dose of 10-80 mg, Q2D.

The dosage regimens (e.g., the dose e.g., the dose based on the body weight or the fixed dose, the frequency of administration) of the substance A and the substance B, may also change over the course of treatment, e.g., depending on the reaction of the subject. For example, in some embodiments, the subsequent dose of a certain substance may be lower than the initial dose.

Combination of a Substance A and a Substance C

In another aspect, the present disclosure provides a pharmaceutical combination comprising a substance A and a substance C;

wherein the substance A is Compound F, a crystal form thereof, a pharmaceutically acceptable salt thereof or a solvate thereof;

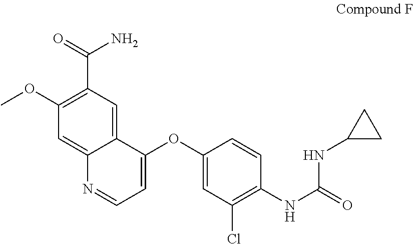 [SMILES: COc1cc2nccc(Oc3ccc(NC(=O)NC4CC4)c(Cl)c3)c2cc1C(N)=O]

the substance C is an antibody N comprising a CDR amino acid sequence selected from the group consisting of SEQ ID NOs: 38, 39, 40, 41, 42, 43, 44, 45, 46, 47, 48, 49, 50 and 51, or an antigen binding fragment thereof.

The pharmaceutical combination as defined above can further comprise a pharmaceutical excipient.

The pharmaceutical combination as defined above can be in the form of a fixed combination or a non-fixed combination. In some embodiments, the pharmaceutical combination is in the form of a non-fixed combination.

In some embodiments, the antibody N may comprise:

(a) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence of SEQ ID NO: 42, a LCDR2 amino acid sequence of SEQ ID NO: 47, and a LCDR3 amino acid sequence of SEQ ID NO: 48;

(b) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 42, a LCDR2 amino acid sequence of SEQ ID NO: 47, and a LCDR3 amino acid sequence of SEQ ID NO: 49;

(c) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 43, a LCDR2 amino acid sequence of SEQ ID NO: 47, and a LCDR3 amino acid sequence of SEQ ID NO: 49;

(d) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 44, a LCDR2 amino acid sequence of SEQ ID NO: 47, and a LCDR3 amino acid sequence of SEQ ID NO: 49;

(e) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 49;

(f) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 44, a LCDR2 amino acid sequence of SEQ ID NO: 47, and a LCDR3 amino acid sequence of SEQ ID NO: 49;

(g) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 49;

(h) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 50;

(i) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 46, a LCDR2 amino acid sequence of SEQ ID NO: 47, and a LCDR3 amino acid sequence of SEQ ID NO: 51; or

(j) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 46, a LCDR2 amino acid sequence of SEQ ID NO: 47, and a LCDR3 amino acid sequence of SEQ ID NO: 48.

In some embodiments, the antibody N may comprise:

(a) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and

(b) a light chain variable region comprising a LCDR1 amino acid sequence selected from the group consisting of SEQ ID NO: 42, a LCDR2 amino acid sequence of SEQ ID NO: 47, and a LCDR3 amino acid sequence of SEQ ID NO: 49.

In some embodiments, the antibody N may comprise:

(a) a heavy chain variable region comprising an amino acid sequence of SEQ ID NO: 29; and a light chain variable region comprising an amino acid sequence of SEQ ID NO: 31;

(b) a heavy chain variable region comprising an amino acid sequence of SEQ ID NO: 30; and a light chain variable region comprising an amino acid sequence of SEQ ID NO: 31;

(c) a heavy chain variable region comprising an amino acid sequence of SEQ ID NO: 30; and a light chain variable region comprising an amino acid sequence of SEQ ID NO: 32;

(d) a heavy chain variable region comprising an amino acid sequence of SEQ ID NO: 30; and a light chain variable region comprising an amino acid sequence of SEQ ID NO: 33;

(e) a heavy chain variable region comprising an amino acid sequence of SEQ ID NO: 29; and a light chain variable region comprising an amino acid sequence of SEQ ID NO: 34;

(f) a heavy chain variable region comprising an amino acid sequence of SEQ ID NO: 29; and a light chain variable region comprising an amino acid sequence of SEQ ID NO: 33;

(g) a heavy chain variable region comprising an amino acid sequence of SEQ ID NO: 30; and a light chain variable region comprising an amino acid sequence of SEQ ID NO: 34;

(h) a heavy chain variable region comprising an amino acid sequence of SEQ ID NO: 30; and a light chain variable region comprising an amino acid sequence of SEQ ID NO: 35;

(i) a heavy chain variable region comprising an amino acid sequence of SEQ ID NO: 29; and a light chain variable region comprising an amino acid sequence of SEQ ID NO: 36; or

(j) a heavy chain variable region comprising an amino acid sequence of SEQ ID NO: 30; and a light chain variable region comprising an amino acid sequence of SEQ ID NO: 37.

In some embodiments, the antibody N may comprise:

(a) a heavy chain variable region comprising an amino acid sequence of SEQ ID NO: 30; and

(b) a light chain variable region comprising an amino acid sequence of SEQ ID NO: 31.

CDRs amino acid sequence of exemplary antibody N, 1H6, 2E5, 2G4, 2C2, A6W, 1G10, 2B1, L1I, 5C4 and 8C10 are shown in the following Table 3.

TABLE 3

| | | SEQ ID | | SEQ ID | | SEQ ID | |
|---|---|---|---|---|---|---|---|
| Clone ID. | | NO | CDR1 | NO | CDR2 | NO | CDR3 |
| 1H6 | Heavy | 38 | TYYIS | 39 | YINMGSGGTNYNEKFKG | 40 | LGYFDY |
| | chain | | | | | | |
| | Light | 42 | RSSQSLLDSDGGTYLY | 47 | LVSTLGS | 48 | MQLTHENYT |
| | chain | | | | | | |
| 2E5 | Heavy | 38 | TYYIS | 39 | YINMGSGGTNYNEKFKG | 41 | IGYEDY |
| | chain | | | | | | |
| | Light | 42 | RSSQSLLDSDGGTYLY | 47 | LVSTLGS | 49 | MQLTHWPYT |
| | chain | | | | | | |
| 2G4 | Heavy | 38 | TYYIS | 39 | YINMGSGGTNYNEKFKG | 41 | IGYEDY |
| | chain | | | | | | |
| | Light | 43 | RSSQSLLDSDGSTYLY | 47 | LVSTLGS | 49 | MQLTHWPYT |
| | chain | | | | | | |
| 2C2 | Heavy | 38 | TYYIS | 39 | YINMGSGGTNYNEKFKG | 41 | IGYEDY |
| | chain | | | | | | |
| | Light | 44 | RSSQSLLDSDGATYLY | 47 | LVSTLGS | 49 | MQLTHWPYT |
| | chain | | | | | | |
| A6W | Heavy | 38 | TYYIS | 39 | YINMGSGGTNYNEKFKG | 40 | LGYFDY |
| | chain | | | | | | |
| | Light | 45 | RSSQSLLDSDGNTYLY | 47 | LVSTLGS | 49 | MQLTHWPYT |
| | chain | | | | | | |
| 1G10 | Heavy | 38 | TYYIS | 39 | YINMGSGGTNYNEKFKG | 40 | LGYFDY |
| | chain | | | | | | |
| | Light | 44 | RSSQSLLDSDGATYLY | 47 | LVSTLGS | 49 | MQLTHWPYT |
| | chain | | | | | | |
| 2B1 | Heavy | 38 | TYYIS | 39 | YINMGSGGTNYNEKFKG | 41 | IGYFDY |
| | chain | | | | | | |
| | Light | 45 | RSSQSLLDSDGNTYLY | 47 | LVSTLGS | 49 | MQLTHWPYT |
| | chain | | | | | | |
| L11 | Heavy | 38 | TYYIS | 39 | YINMGSGGTNYNEKFKG | 41 | IGYFDY |
| | chain | | | | | | |
| | Light | 45 | RSSQSLLDSDGNTYLY | 47 | LVSTLGS | 50 | MQLTHAPYT |
| | chain | | | | | | |
| 5C4 | Heavy | 38 | TYYIS | 39 | YINMGSGGTNYNEKFKG | 40 | LGYFDY |
| | chain | | | | | | |
| | Light | 46 | RSSQSLLDSDGQTYLY | 47 | LVSTLGS | 51 | MQLTHEPYT |
| | chain | | | | | | |
| 8C10 | Heavy | 38 | TYYIS | 39 | YINMGSGGTNYNEKFKG | 40 | LGYFDY |
| | chain | | | | | | |
| | Light | 46 | RSSQSLLDSDGQTYLY | 47 | LVSTLGS | 48 | MQLTHENYT |
| | chain | | | | | | |

Exemplary antibody N, 1H6, 2E5, 2G4, 2C2, A6W, 1G10, 2B1, L1I, 5C4 and 8C10 have heavy chain variable region and light chain variable region as shown in the following Table 4, and a constant region of human IgG4 isotype.

TABLE 4

| Clone ID | | SEQ ID NO | Amino acid sequence |
|---|---|---|---|
| 1H6 | Heavy chain | 29 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | LGYFDYWGQGTMVTVSS |
| | Light chain | 31 | DVVMTQSPLSLPVTLGQPASISCRSSQSLLDSDG |
| | | | GTYLYWFQQRPGQSPRRLIYLVSTLGSGVPDRF |
| | | | SGSGSGTDFTLKISRVEAEDVGVYYCMQLTHW |
| | | | PYTFGQGTKLEIK |
| 2E5 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 31 | DVVMTQSPLSLPVTLGQPASISCRSSQSLLDSDG |
| | | | GTYLYWFQQRPGQSPRRLIYLVSTLGSGVPDRF |
| | | | SGSGSGTDFTLKISRVEAEDVGVYYCMQLTHW |
| | | | PYTFGQGTKLEIK |
| 2G4 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 32 | DVVMTQSPLSLPVTLGQPASISCRSSQSLLDSDG |
| | | | STYLYWFQQRPGQSPRRLIYLVSTLGSGVPDRFS |
| | | | GSGSGTDFTLKISRVEAEDVGVYYCMQLTHWP |
| | | | YTFGQGTKLEIK |
| 2C2 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 33 | DVVMTQSPLSLPVTLGQPASISCRSSQSLLDSDG |
| | | | ATYLYWFQQRPGQSPRRLIYLVSTLGSGVPDRFS |
| | | | GSGSGTDFTLKISRVEAEDVGVYYCMQLTHWP |
| | | | YTFGQGTKLEIK |
| A6W | Heavy chain | 29 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | LGYFDYWGQGTMVTVSS |
| | Light chain | 34 | DVVMTQSPLSLPVTLGQPASISCRSSQSLLDSDG |
| | | | NTYLYWFQQRPGQSPRRLIYLVSTLGSGVPDRF |
| | | | SGSGSGTDFTLKISRVEAEDVGVYYCMQLTHW |
| | | | PYTFGQGTKLEIK |
| 1G10 | Heavy chain | 29 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | LGYFDYWGQGTMVTVSS |
| | Light chain | 33 | DVVMTQSPLSLPVTLGQPASISCRSSQSLLDSDG |
| | | | ATYLYWFQQRPGQSPRRLIYLVSTLGSGVPDRFS |
| | | | GSGSGTDFTLKISRVEAEDVGVYYCMQLTHWP |
| | | | YTFGQGTKLEIK |
| 2B1 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 34 | DVVMTQSPLSLPVTLGQPASISCRSSQSLLDSDG |
| | | | NTYLYWFQQRPGQSPRRLIYLVSTLGSGVPDRF |
| | | | SGSGSGTDFTLKISRVEAEDVGVYYCMQLTHW |
| | | | PYTFGQGTKLEIK |
| L11 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 35 | DVVMTQSPLSLPVTLGQPASISCRSSQSLLDSDG |
| | | | NTYLYWFQQRPGQSPRRLIYLVSTLGSGVPDRF |
| | | | SGSGSGTDFTLKISRVEAEDVGVYYCMQLTHAP |
| | | | YTFGQGTKLEIK |
| 5C4 | Heavy chain | 29 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | LGYFDYWGQGTMVTVSS |
| | Light chain | 36 | DVVMTQSPLSLPVTLGQPASISCRSSQSLLDSDG |
| | | | QTYLYWFQQRPGQSPRRLIYLVSTLGSGVPDRF |
| | | | SGSGSGTDFTLKISRVEAEDVGVYYCMQLTHEP |
| | | | YTFGQGTKLEIK |
| 8C10 | Heavy chain | 30 | QVQLVQSGAEVKKPGSSVKVSCKASGFTFTTY |
| | | | YISWVRQAPGQGLEYLGYINMGSGGTNYNEKF |
| | | | KGRVTITADKSTSTAYMELSSLRSEDTAVYYCAI |
| | | | IGYFDYWGQGTMVTVSS |
| | Light chain | 37 | DVVMTQSPLSLPVTLGQPASISCRSSQSLLDSDG |
| | | | QTYLYWFQQRPGQSPRRLIYLVSTLGSGVPDRF |
| | | | SGSGSGTDFTLKISRVEAEDVGVYYCMQLTHEN |
| | | | YTFGQGTKLEIK |

In some embodiments, the antibody N can be anti-PD-1 antibody.

In some embodiments, the antibody N can be capable of specifically binding to human PD-1.

In some embodiments, the antibody N can be a monoclonal antibody, fully human antibody, humanized antibody, chimeric antibody, recombinant antibody, bispecific antibody, labeled antibody, bivalent antibody, or anti-idiotypic antibody. In some embodiments, the antibody N can be humanized antibody.

In some embodiments, the antibody N can be a camelized single domain antibody, a diabody, a scFv, an scFv dimer, a BsFv, a dsFv, a (dsFv)₂, a dsFv-dsFv′, an Fv fragment, a Fab, a Fab′, a F(ab′)₂, a ds diabody, a nanobody, a domain antibody, or a bivalent domain antibody.

In some embodiments, the antibody N may further comprise an immunoglobulin (e.g., human IgG4) constant region. In some embodiments, the antibody N may further comprise a heavy chain constant region of human IgG4 (e.g., IgG4 isotype), and a light chain constant region of human λ or κ light chain.

Pharmaceutical Composition, Kit and Use of the Substance A and the Substance C in Combination

In another aspect, the present disclosure also provides a pharmaceutical composition comprising a substance A and a substance C; wherein the substance A and the substance C are as defined above.

The pharmaceutical composition may further comprise a pharmaceutical excipient.

In another aspect, the present disclosure also provides a kit comprising:

a first container comprising a first pharmaceutical composition comprising a substance A; and

a second container comprising a second pharmaceutical composition comprising a substance C;

wherein the substance A and the substance C are as defined above.

The first pharmaceutical composition may further comprise a pharmaceutical excipient.

The second pharmaceutical composition may further comprise a pharmaceutical excipient.

In another aspect, the present disclosure also provides a use of the pharmaceutical combination or the pharmaceutical composition as defined above in manufacturing a medicament for treating a cancer.

In another aspect, the present disclosure also provides a method for treating a cancer comprising administering (e.g., a therapeutically effective amount of) the pharmaceutical combination or the pharmaceutical composition as defined above (e.g., to a subject (e.g., a human or a mouse) in need thereof).

In some embodiments, the cancer can be non-small cell lung cancer, small cell lung cancer, renal cell cancer, colorectal cancer, colon cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas or myelomas.

In some embodiments, the cancer can be colon cancer.

Dosages Regimens of the Substance A and the Substance C in Combination

The substance A and the substance C can be administrated simultaneously or separately.

In some embodiments, the substance A and the substance C can be administrated separately.

The term “administrated simultaneously” means administration at a same time point. The substance A and the substance C can be present in a single pharmaceutical composition (e.g., in a single dosage form, e.g., in one capsule) to be administrated at a same time point; or, the substance A and the substance C can also be present separately in different pharmaceutical compositions to be administrated at a same time point.

The term “administrated separately” means administration at different time points. For example, the substance A and the substance C can be present separately in different pharmaceutical compositions to be administrated at different time points. The separated administration may be close in time or distant in time but make sure the substance A and the substance C can act in concert so as to provide the desired therapeutic effect. For example, the substance A can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the substance C, to a subject in need thereof. In some embodiments, the substance A and the substance C are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In some embodiments, the substance A and the substance C are administered 30 minutes to 1 hour apart.

Whether the substance A and the substance C are administrated simultaneously or separately, the dosage regimens (e.g., route, dose and frequency of administration) of the substance A and the substance C can be the same or different, which can be adjusted by a person skilled in the art in order to provide an optimal therapeutic effect as needed.

Suitable routes of administration for the substance A and the substance C includes gastrointestinal administration (e.g., oral administration) and parenteral administration (e.g., injection, e.g., intraperitoneal injection, intravenous injection, subcutaneous injection, or intramuscular injection).

Oral administration may involve swallowing, so that the substance enters the gastrointestinal tract, and/or buccal, lingual, or sublingual administration by which the substance enters the blood stream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches. Further, the substance can be administered as a spray dried dispersion. Solid formulations for oral administration may be formulated to be immediate and/or modified release. Modified release formulations include delayed-, sustained-, pulsed-, controlled-, targeted and programmed release. Liquid formulations include suspensions, solutions, syrups and elixirs. Such formulations may be employed as fillers in soft or hard capsules (made, for example, from gelatin or hydroxypropyl methylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques. Formulations for parenteral administration may be formulated to be immediate and/or modified release. Modified release formulations include delayed-, sustained-, pulsed-, controlled-, targeted and programmed release.

In some embodiments, the substance A, e.g., the mesylate of Compound F, can be administrated orally.

In some embodiments, the substance B, e.g., the antibody N, can be administrated by injection, e.g., intraperitoneal injection, intravenous injection, subcutaneous injection, or intramuscular injection, e.g., intraperitoneal injection.

The substance A can be administered (e.g., orally) at a dose based on the body weight of a subject, non-limiting examples of the dose can range from 0.01 to 50 mg/kg, e.g., 0.05 mg/kg, 0.1 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 25 mg/kg, 30 mg/kg, 35 mg/kg, 40 mg/kg, 45 mg/kg or 50 mg/kg (e.g., based on the mesylate of Compound F). In some embodiments, the substance A (e.g., the mesylate of Compound F) is administered at a dose of 1-10 mg/kg, e.g., 1 mg/kg. The above dose of the substance A can be administered to a subject in a frequency of QD (once a day), BID (twice a day), TID (three times a day), Q2D (once every two days), QW (once a week), BIW (twice a week) or Q2W (once every two weeks). In some embodiments, the above dose of the substance A (e.g., the mesylate of Compound F) is administrated in a frequency of QD.

In some embodiments, the substance A, e.g., the mesylate of Compound F, is administrated (e.g., orally) to a subject (e.g., a human or a mouse) at a dose of 1 mg/kg, QD.

The substance A can also be administered (e.g., orally) to a subject in a fixed (e.g., predetermined) dose. Non-limiting examples of the fixed dose can range from 0.1-1000 mg, e.g., 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mg (e.g., based on the mesylate of Compound F). In some embodiments, the substance A (e.g., the mesylate of Compound F) is administered at a dose of 10-30 mg, e.g., 20 mg.

The above fixed dose of the substance A can be administrated to the subject in a frequency of QD (once a day), BID (twice a day), TID (three times a day), Q2D (once every two days), Q3D (once every three days), Q4D (once every four days), QW (once a week), BIW (twice a week) or Q2W (once every two weeks). In some embodiments, the above fixed doses of the substance A (e.g., the mesylate of Compound F) is administrated in a frequency of QD.

In some embodiments, the substance A, e.g., the mesylate of Compound F, is administrated (e.g., orally) to a subject (e.g., a human or a mouse) at a dose of 10-30 mg, QD.

The substance C can be administered (e.g., by injection, e.g., intraperitoneal injection, intravenous injection, subcutaneous injection, or intramuscular injection, e.g., intraperitoneal injection) at a dose based on the body weight of a subject, non-limiting examples of the dose can range from 0.01 to 50 mg/kg, e.g., 0.05 mg/kg, 0.1 mg/kg, 0.2 mg/kg, 0.25 mg/kg, 0.3 mg/kg, 0.35 mg/kg, 0.4 mg/kg, 0.45 mg/kg, 0.5 mg/kg, 0.55 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 25 mg/kg, 30 mg/kg, 35 mg/kg, 40 mg/kg, 45 mg/kg or 50 mg/kg (e.g., based on the mesylate of Compound F). In some embodiments, the substance C is administered at a dose of 1-10 mg/kg, e.g., 1-2 mg/kg. The above dose of the substance C can be administered to a subject in a frequency of QD (once a day), BID (twice a day), TID (three times a day), Q2D (once every two days), Q3D (once every three days), Q4D (once every four days), QW (once a week), BIW (twice a week) or Q2W (once every two weeks). In some embodiments, the above dose of the substance C is administrated in a frequency of Q2D, Q3D or Q4D.

In some embodiments, the substance C can be administrated (e.g., by injection, e.g., intraperitoneal injection, intravenous injection, subcutaneous injection, or intramuscular injection, e.g., intraperitoneal injection) to a subject (e.g., a human or a mouse) at a dose of 1-2 mg/kg, Q2D, Q3D or Q4D.

The substance C can also be administered (e.g., by injection, e.g., intraperitoneal injection, intravenous injection, subcutaneous injection, or intramuscular injection, e.g., intraperitoneal injection) to a subject in a fixed (e.g., predetermined) dose. Non-limiting examples of the fixed dose can range from 0.1-1000 mg, e.g., 0.1, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475 or 500 mg. In some embodiments, the substance C is administered at a dose of 10-80 mg. The above fixed dose of the substance C can be administrated to the subject in a frequency of QD (once a day), BID (twice a day), TID (three times a day), Q2D (once every two days), Q3D (once every three days), Q4D (once every four days), QW (once a week), BIW (twice a week) or Q2W (once every two weeks). In some embodiments, the above fixed doses of the substance C is administrated in a frequency of Q2D, Q3D or Q4D.

In some embodiments, the substance C can be administrated (e.g., by injection, e.g., intraperitoneal injection, intravenous injection, subcutaneous injection, or intramuscular injection, e.g., intraperitoneal injection) to a subject (e.g., a human or a mouse) at a dose of 10-80 mg, Q2D, Q3D or Q4D.

The dosage regimens (e.g., the dose, e.g., the dose based on the body weight or the fixed dose, the frequency of administration) of the substance A and the substance C, may also change over the course of treatment, e.g., depending on the reaction of the subject. For example, in some embodiments, the subsequent dose of a certain substance may be lower than the initial dose.

The following description of the disclosure is merely intended to illustrate various embodiments of the disclosure. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included by the invention. All references cited herein, including publications, patents and patent applications are incorporated herein by reference in their entireties.

### Definitions

The antibody M (e.g., antibody 1.4.1, 1.14.4, 1.20.15, or 1.46.11) or an antigen binding fragment thereof as described herein is known can be found in WO2017020858A1 and CN106432501A, which are incorporated herein by reference in their entireties.

The antibody N (e.g., antibody 1H6, 2E5, 2G4, 2C2, A6W, 1G10, 2B1, L1I, 5C4 and 8C10) or an antigen binding fragment thereof as described herein can be found in WO2018053709A1 and CN107840887A, which are incorporated herein by reference in their entireties.

The Compound F is known as Lenvatinib, CAS Number 417716-92-8. The Compound F and its derivatives, e.g., salts, crystal forms, solvates, are all within the scope of the present disclosure, which can be found in references, e.g., WO02/32872, WO2005/063713, WO2011/021597, WO2016/140717, all of which are incorporated herein by reference in their entireties.

The term “pharmaceutical combination” is used herein to mean a product including the active ingredients (e.g. the antibody M, the antibody N, the Compound F) according to the present disclosure. The active ingredients included by the pharmaceutical combination can be present in a single entity (e.g., a single dosage form, e.g., in one injection, in one tablet or in one capsule), and thus can be administered to a subject simultaneously. The active ingredients included by the pharmaceutical combination can also be present in separate entities (e.g., one active ingredient is present in an tablet, while the other active ingredient is present in a capsule), and thus can be administered to a subject independently of each other, either simultaneously or separately with no specific time limits. If the active ingredients included by the pharmaceutical combination are present in separate entities, they can be sold independently of each other and just instruction of the possibility of their combined use is provided in the package equipment, e.g., leaflet or the like, or in other information, e.g., provided to physicians and medical staff (e.g., oral communications).

The term “fixed combination” means that the combination partners are present in the form of a single entity (e.g., a single dosage form, e.g., in one injection, in one tablet or in one capsule), and can be administered to a subject simultaneously.

The term “non-fixed combination” means that the combination partners are present as separate entities, and can be administered to a subject independently of each other, either simultaneously or separately with no specific time limits. The combination partners may be used as entirely separate pharmaceutical dosage forms or pharmaceutical formulations, and can be are also sold independently of each other and just instruction of the possibility of their combined use is provided in the package equipment, e.g., leaflet or the like, or in other information, e.g., provided to physicians and medical staff (e.g., oral communications).

The term “antibody” as used herein includes any immunoglobulin, monoclonal antibody, polyclonal antibody, multispecific antibody, or bispecific (bivalent) antibody that binds to a specific antigen. A native intact antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variables region in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196, 901 (1987); Chothia, C. et al., Nature. December 21-28; 342 (6252): 877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “antigen-binding fragment” as used herein refers to an antibody fragment formed from a portion of an antibody comprising one or more CDRs, or any other antibody fragment that binds to an antigen but does not comprise an intact native antibody structure. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding fragment is capable of binding to the same antigen to which the parent antibody binds. In some embodiments, an antigen-binding fragment may comprise one or more CDRs from a particular human antibody grafted to a framework region from one or more different human antibodies.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region.

“F(ab′)₂” refers to a dimer of Fab′.

“Fc” with regard to an antibody refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)).

“Single-chain Fv-Fc antibody” or “scFv-Fc” refers to an engineered antibody consisting of a scFv connected to the Fc region of an antibody.

“Camelized single domain antibody,” “heavy chain antibody,” or “HCAb” refers to an antibody that contains two V_{H} domains and no light chains (Riechmann L. and Muyldermans S., J Immunol Methods. December 10; 231 (1-2): 25-38 (1999); Muyldermans S., J Biotechnol. June; 74(4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363 (6428): 446-8 (1993); Nguyen V K. et al. “Heavy-chain antibodies in Camelidae; a case of evolutionary innovation,” Immunogenetics. April; 54(1): 39-47 (2002); Nguyen V K. et al. Immunology. May; 109(1): 93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)).

A “nanobody” refers to an antibody fragment that consists of a VHH domain from a heavy chain antibody and two constant domains, CH2 and CH3.

“Diabodies” include small antibody fragments with two antigen-binding sites, wherein the fragments comprise a V_{H} domain connected to a V_{L} domain in the same polypeptide chain (V_{H}-V_{L} or V_{H}-V_{L}) (see, e.g., Holliger P. et al., Proc Natl Acad Sci USA. July 15; 90(14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain instances, two or more V_{H} domains are covalently joined with a peptide linker to create a bivalent or multivalent domain antibody. The two V_{H} domains of a bivalent domain antibody may target the same or different antigens.

In some embodiments, a “(dsFv)₂” comprises three peptide chains: two V_{H} moieties linked by a peptide linker and bound by disulfide bridges to two V_{L} moieties.

In some embodiments, a “bispecific ds diabody” comprises V_{H1}-V_{L2} (linked by a peptide linker) bound to V_{L1}-V_{H2} (also linked by a peptide linker) via a disulfide bridge between V_{H1} and V_{L1}.

In some embodiments, a “bispecific dsFv” or dsFv-dsFv′” comprises three peptide chains: a V_{H1}-V_{H2} moiety wherein the heavy chains are linked by a peptide linker (e.g., a long flexible linker) and bound to V_{L1} and V_{L2} moieties, respectively, via disulfide bridges, wherein each disulfide paired heavy and light chain has a different antigen specificity.

In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'S of the other moiety and form two binding sites which can target the same antigens (or eptipoes) or different antigens (or eptipoes). In other embodiments, an “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

The term “fully human” as used herein, with reference to antibody or antigen-binding fragment, means that the antibody or the antigen-binding fragment has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

The term “humanized” as used herein, with reference to antibody or antigen-binding fragment, means that the antibody or the antigen-binding fragment comprises CDRs derived from non-human animals, FR regions derived from human, and when applicable, the constant regions derived from human. A humanized antibody or antigen-binding fragment is useful as human therapeutics in certain embodiments because it has reduced immunogenicity in human. In some embodiments, the non-human animal is a mammal, for example, a mouse, a rat, a rabbit, a goat, a sheep, a guinea pig, or a hamster. In some embodiments, the humanized antibody is composed of substantially all human sequences except for the CDR sequences which are non-human. In some embodiments, the FR regions derived from human may comprise the same amino acid sequence as the human antibody from which it is derived, or it may comprise some amino acid changes, for example, no more than 10, 9, 8, 7, 6, 5, 4, 3, 2, or 1 changes of amino acid. In some embodiments, such change in amino acid could be present in heavy chain FR regions only, in light chain FR regions only, or in both chains. In some preferable embodiments, the humanized antibodies comprise human FR1-3 and human JH and Jκ.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human species, such as from mouse.

“PD-L1” as used herein refers to programmed cell death ligand 1 (PD-L1, see, for example, Freeman et al. (2000) J. Exp. Med. 192: 1027). Representative amino acid sequence of human PD-L1 is disclosed under the NCBI accession number: NP_054862.1, and the representative nucleic acid sequence encoding the human PD-L1 is shown under the NCBI accession number: NM_014143.3. PD-L1 is expressed in placenta, spleen, lymph nodes, thymus, heart, fetal liver, and is also found on many tumor or cancer cells. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

“Anti-PD-L1 antibody” as used herein refers to an antibody that is capable of specifically binding to PD-L1 (e.g., human or monkey PD-L1) with an affinity which is sufficient to provide for diagnostic and/or therapeutic use.

“Anti-PD-1 antibody” as used herein refers to an antibody that is capable of specifically binding to PD-1 (e.g., human or monkey PD-1) with an affinity which is sufficient to provide for diagnostic and/or therapeutic use.

The term “specific binding” or “specifically binds” as used herein refers to a non-random binding reaction between two molecules, such as for example between an antibody and an antigen. K_{D} as used herein refers to the ratio of the dissociation rate to the association rate (k_{off}/kₒₙ), may be determined using surface plasmon resonance methods for example using instrument such as Biacore.

The term “epitope” as used herein refers to the specific group of atoms or amino acids on an antigen to which an antibody binds. Two antibodies may bind the same epitope within an antigen if they exhibit competitive binding for the antigen. For example, if an antibody or antigen-binding fragment as disclosed herein blocks binding of the exemplary antibodies such as 1.4.1, 1.14.4, 1.20.15, and 1.46.11 to human PD-L1, then the antibody or antigen-binding fragment may be considered to bind the same epitope as those exemplary antibodies.

A particular amino acid residue within the epitope can be mutated, e.g. by alanine scanning mutagenesis, and mutations that reduce or prevent protein binding are identified. An “alanine scanning mutagenesis” is a method that can be performed for identifying certain residues or regions of a protein that affect the interaction of the epitope with another compound or protein that binds to it. A residue or group of target residues within the protein is replaced by a neutral or negatively charged amino acid (most preferably alanine or polyalanine, or a conservative amino acid substitution). Any mutation of the amino acid residues or codons encoding the same that reduces binding of the protein more than a threshold or reduces binding of the protein to the maximal degree than other mutations is likely to be within the epitope bound by the protein. In some embodiments of the present disclosure, the epitope that is critical for the PD-L1 antibody comprises at least one of the amino acid residues of E58, E60, D61, K62, N63 and R113.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al, J. Mol. Biol., 215:403-410 (1990); Stephen F. et al, Nucleic Acids Res., 25:3389-3402 (1997)), ClustalW2 (available on the website of European Bioinformatics Institute, see also, Higgins D. G. et al, Methods in Enzymology, 266:383-402 (1996); Larkin M. A. et al, Bioinformatics (Oxford, England), 23(21): 2947-8 (2007)), and ALIGN or Megalign (DNASTAR) software. Those skilled in the art may use the default parameters provided by the tool, or may customize the parameters as appropriate for the alignment, such as for example, by selecting a suitable algorithm.

“T cell” as used herein includes CD4⁺ T cells, CD8⁺ T cells, T helper 1 type T cells, T helper 2 type T cells, T helper 17 type T cells and inhibitory T cells.

“Effector functions” as used herein refer to biological activities attributable to the binding of Fc region of an antibody to its effectors such as C1 complex and Fc receptor. Exemplary effector functions include: complement dependent cytotoxicity (CDC) induced by interaction of antibodies and C1q on the C1 complex; antibody-dependent cell-mediated cytotoxicity (ADCC) induced by binding of Fc region of an antibody to Fc receptor on an effector cell; and phagocytosis.

“Cancer” as used herein refers to any medical condition mediated by neoplastic or malignant cell growth, proliferation, or metastasis, and includes both solid cancers and non-solid cancers such as leukemia. “Tumor” as used herein refers to a solid mass of neoplastic and/or malignant cells.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. With regard to cancer, “treating” or “treatment” may refer to inhibiting or slowing neoplastic or malignant cell growth, proliferation, or metastasis, preventing or delaying the development of neoplastic or malignant cell growth, proliferation, or metastasis, or some combination thereof. With regard to a tumor, “treating” or “treatment” includes eradicating all or part of a tumor, inhibiting or slowing tumor growth and metastasis, preventing or delaying the development of a tumor, or some combination thereof.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated”, but the same polynucleotide or polypeptide is “isolated” if it has been sufficiently separated from the coexisting materials of its natural state so as to exist in a substantially pure state. In some embodiments, the antibodies and antigen-binding fragments are isolated, and can have a purity of at least 90%, 93%, 95%, 96%, 97%, 98%, 99% as determined by conventional methods such as electrophoretic methods (such as SDS-PAGE, isoelectric focusing, capillary electrophoresis), or chromatographic methods (such as ion exchange chromatography or reverse phase HPLC).

The present disclosure includes all pharmaceutically acceptable isotopically-labeled substances of the disclosure, e.g., the Compound F, antibody M, antibody N, wherein one or more atoms are replaced by atoms having the same atomic number, but an atomic mass or mass number different from the atomic mass or mass number usually found in nature. Examples of isotopes suitable for inclusion in the substances of the disclosure comprises isotopes of hydrogen, such as ²H and ³H; carbon, such as ¹¹C, ¹³C and ¹⁴C; nitrogen, such as ¹³N and ¹⁵N; oxygen, such as ¹⁵O, ¹⁷O and ¹⁸O.

Certain derivatives that can be converted into the substance (e.g., the Compound F) of the disclosure when administered into the body are also within the scope of the disclosure. Such derivatives are referred to as “prodrug”. Further information on the use of prodrug may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

The term “solvate” as used herein refers to a complex formed by the combination of a compound and a stoichiometric or non-stoichiometric solvent. The solvent molecule(s) in the solvate can be present in an ordered or non-ordered arrangement. Examples of the solvents include, but are not limited to, water, methanol, ethanol and the like.

The term “pharmaceutically acceptable salt” as used herein refers to a salt formed by a compound and a relatively non-toxic and pharmaceutically acceptable acid or base. When a compound contains a relatively acidic functional group, a base addition salt can be obtained by contacting a sufficient amount of a pharmaceutically acceptable base with the neutral form of the compound in a pure solution or a suitable inert solvent. Examples of pharmaceutically acceptable base addition salts include, but are not limited to, lithium salts, sodium salts, potassium salts, calcium salts, aluminum salts, magnesium salts, zinc salts, bismuth salt, ammonium salts and diethanolamine salts. When a compound contains a relatively basic functional group, an acid addition salt can be obtained by contacting the neutral form of the compound with a sufficient amount of a pharmaceutically acceptable acid in a pure solution or a suitable inert solvent. Examples of pharmaceutically acceptable acids include inorganic acids, wherein the inorganic acids include, but are not limited to, hydrochloric acid, hydrobromic acid, hydroiodic acid, nitric acid, carbonic acid, bicarbonate, phosphoric acid, phosphorous acid, sulfuric acid and the like. Examples of pharmaceutically acceptable acid include organic acids, wherein the organic acids include, but are not limited to, acetic acid, propionic acid, oxalic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, salicylic acid, tartaric acid, methanesulfonic acid, isonicotinic acid, acidic citric acid, oleic acid, tannic acid, pantothenic acid, ascorbic acid, gentisic acid, fumaric acid, gluconic acid, formic acid, ethanesulfonic acid, pamoic acid (i.e., 4,4′-methylenebis (3-hydroxy-2-naphthoic acid)), amino acids (such as glutamic acid, arginine) and the like. When a compound contains both a relatively basic functional group and a relatively acidic functional group, it can be converted to a base addition salt or an acid addition salt. The pharmaceutically acceptable salt can be referred to Berge et al., “Pharmaceutical Salts”, Journal of Pharmaceutical Science 66: 1-19 (1977) or Handbook of Pharmaceutical Salts: Properties, Selection, and Use (P. Heinrich Stahl and Camille G. Wermuth, ed., Wiley-VCH, 2002).

The term “pharmaceutical excipient” as used herein refers to an excipient and/or an additive used in producing a medicament or in formulating a formulation, which includes all substances contained in a pharmaceutical preparation except the active ingredient. Examples of the pharmaceutical excipient can refer to Pharmacopoeia of the People's Republic of China (Volume IV, 2015 edition), or the Handbook of Pharmaceutical Excipients (Raymond C Rowe, 2009 Sixth Edition).

A “therapeutically effective amount” of a substance, or a pharmaceutical composition, or a pharmaceutical combination, refers to an amount sufficient to cure, alleviate or partially arrest the clinical manifestations of a given disease or disorder and its complications. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or clinical scientist. It will be appreciated that the unit content of each active agent contained in an individual dose of each dosage form need not in itself constitute an effective amount since the necessary effective amount can be reached by administration of a plurality of dosage units.

The term “subject” as used herein refers to any animal that is to be administered or has been administered the compound or composition in accordance with an embodiment of the disclosure, which is preferably a mammal, more preferably a human. The term “mammal” as used herein includes any mammal. Examples of mammals include, but are not limited to, cows, horses, sheep, pigs, cats, dogs, mice, rats, rabbits, guinea pigs, monkeys and humans. The preferred subject is a human.

The term “in need thereof” would be a subject known or suspected of having or being at risk of developing a disease.

The term “container” as used herein refers to any container suitable for storage, transport, distribution and/or disposal of a pharmaceutical product.

The abbreviations “p.o” (i.e., orally), “i.p.” (e.g., intraperitoneal injection), “q.d” or “QD” (i.e. daily), “q2d” or “Q2D” (i.e., once every two days) and the like are used to describe the dosage regiment in their general meanings.

The terms “first”, “second” and the like are only used to distinguish different entities, and are not intended to limit the scope of the disclosure. For example, a substance X and a substance Y are comprised in a first pharmaceutical composition and a second pharmaceutical composition respectively, it means the substance X and the substance Y are separately present in different pharmaceutical compositions.

The use of the terms “a”, “an”, “the”, and similar referents in the context of describing the disclosure are to be construed to cover both the singular and the plural, unless otherwise indicated. The use of any and all examples, or exemplary language (including “e.g.”, “such as” and “for example”) provided herein, is intended to better illustrate the disclosure and is not a limitation on the scope of the disclosure unless otherwise claimed. No language in the specification should be construed as indicating any non-claimed element as essential to the practice of the disclosure.

Without violating the common sense in the art, the above preferred conditions can be arbitrarily combined, then preferred embodiments of the invention are obtained.

The reagents used herein are commercially available.

It has surprisingly been found that, a combination of Lenvatinib mesylate and antibody 1.14.4 achieves greater therapeutic effect than Lenvatinib mesylate and antibody 1.14.4 alone on colon cancer animal model. A combination of Lenvatinib mesylate and antibody 2E5 also achieves greater therapeutic effect than Lenvatinib mesylate and antibody 2E5 alone on colon cancer animal model.

### DETAILED DESCRIPTION OF THE PREFERRED EMBODIMENT

The following examples further illustrate the invention, but the invention is not limited thereto.

### Embodiment 1: Pharmacodynamic Study of Antibody 1.14.4 Monotherapy or in Combination with Lenvatinib Mesylate on B-hPD-1 Humanized Mouse MC38-hPD-L1 Colon Cancer Animal Model

1. Objective of the Study

The objective of the study was to evaluate the therapeutic efficacy of antibody 1.14.4 monotherapy or in combination with Lenvatinib mesylate in the treatment of subcutaneous MC38-hPD-L1 transplanted tumor.

2. Reagents and Equipment

TABLE 5
Reagents and equipment

| Reagents | | | | | |
|---|---|---|---|---|---|
| or | | | Storage | | Physical |
| equipment | Supplier | Art. No | condition | Lot. No | form |
| hIgG4 | CROWN | C0004-4 | — | AB170091 | 5.1 mg/mL |
| | BIO- | | | | solution in |
| | SCIENCE | | | | physio- |
| | INC | | | | logical |
| | TAICANG | | | | saline |
| Lenvatinib | Eisai Co., | — | — | 164H0501 | powder |
| mesylate | Ltd. | | | | |
| DMEM | Cellgro | 10-013- | 4° C. | — | — |
| | | CVR | | | |
| FBS | Excell | FND500- | −20° C. | — | — |
| | | 120820 | | | |
| Vernier | Hexagon | 00534220 | — | — | — |
| caliper | Metrology | | | | |
| Physio- | — | — | — | — | — |
| logical | | | | | |
| saline | | | | | |

3. Test Sample

Antibody 1.14.4 was prepared according to WO2017020858A1 (Examples 1-3).

4. Formulation

4.1. Formulation of hIgG4

5.1 mg/mL solution of hIgG4 in physiological saline was diluted with physiological saline to a concentration of 0.3 mg/mL for use. The prepared solution was stored at 2-8° C. when not in use, and allowed to stand to room temperature before use.

4.2. Formulation of Different Concentration of Lenvatinib Mesylate

Lenvatinib mesylate powder was dissolved in 3 mmol/L HCl solution to prepare 3 mg/mL stock solution of Lenvatinib mesylate. The stock solution was stored at 4° C. when not in use.

1 mg/mL Lenvatinib mesylate solution was prepared by diluting 2.24 mL of 3 mg/mL stock solution with 4.48 mL of 3 mmol/L HCl solution. The prepared solution was stored at 2-8° C. when not in use, and allowed to stand to room temperature before use.

0.3 mg/mL Lenvatinib mesylate solution was prepared by diluting 1.92 mL of 1 mg/mL Lenvatinib mesylate solution with 4.48 mL of 3 mmol/L HCl solution. The prepared solution was stored at 2-8° C. when not in use, and allowed to stand to room temperature before use.

0.1 mg/mL Lenvatinib mesylate solution was prepared by diluting 1.6 mL of 0.3 mg/mL Lenvatinib mesylate solution with 3.2 mL of 3 mmol/L HCl solution. The prepared solution was stored at 2-8° C. when not in use, and allowed to stand to room temperature before use.

4.3. Formulation of Antibody 1.14.4

Antibody 1.14.4 was diluted with physiological saline to prepare 0.3 mg/mL antibody 1.14.4 solution for use. The prepared solution was stored at 2-8° C. when not in use, and allowed to stand to room temperature before use.

5. Animals

Species: Mus musculus;

Strain: C57BL/6;

Name: B-hPD-1 mice;

Sex: male;

Body weight: 17-21 g

Age: 7 weeks;

Number of animals: 85 (56 plus 29 spare) mice;

Animal supplier: Biocytogen Jiangsu Co., Ltd;

License number: SCXK (Jiangsu) 2016-0004;

Animal Certificate No.: 201727553.

6. Animal Housing and Care

Animals were housed in the SPF animal laboratory of Experiment Animal Center in Biocytogen Beijing Co., Ltd using individually ventilated cages (IVC). Animals were acclimated to the environment for three days prior to initiate the study.

Temperature: 20-26° C.;

Humidity: 40%-70%;

Light: 12 hours on and 12 hours off;

Cage: Cage was made of PPSU with a volume of 320 mm×200 mm×135 mm. Corn cobs were used for animal bedding, which were sterilized by autoclave before use. Beddings were changed once a week. Each cage was identified by a cage card marking the number of animals, gender, strain, receiving date, group number, and starting date of the experiment.

Diet and water: Reverse osmosis (RO) water was autoclaved before use. Animals had free access to SPF mouse feed and sterile drinking water.

Animal identification: Each mice was identified by ear tag.

7. Experimental Procedure

7.1. Cell Culture

Mouse colon cancer cells MC38 were purchased from Shun Ran (Shanghai) Biotechnology Co., Ltd., and the cells were cultured in a 37° C., 5% CO₂ incubator supplemented with Dulbecco's Modified Eagle's medium containing 10% inactivated fetal bovine serum. MC38 cells were genetically modified to overexpress human PD-L1 and knock out murine PD-L1, the modified cells were named as MC38-hPD-L1 cells.

7.2. Tumor Inoculation, Group and Dosage Regimen

MC38-hPD-L1 tumor cells were resuspended in 1×PBS to a concentration of 5×10⁶ cells/mL. Each of the B-hPD-1 humanized mice was subcutaneously inoculated at the right lateral thorax region with 0.1 mL of the cell suspension, and a total of 85 mice were inoculated. On the 7th day after inoculation, when the average tumor volume reached about 103 mm³, the mice with moderate tumor volume were selected and randomly divided into 8 groups according to tumor volume, with 7 mice in each group, and the treatment was initiated on the same day. The dosage regimen was shown in Table 6.

TABLE 6
Dosage regimen

| | Treatment | |
|---|---|---|
| Group | Dose^{a}, frequency^{b}, route^{c} | Animals No. |
| G1 | hIgG4, 3 mg/kg, Q2D, i.p. | 7 |
| G2 | Antibody 1.14.4, 3 mg/kg, Q2D, i.p. | 7 |
| G3 | Lenvatinib mesylate, 1 mg/kg, QD, p.o. | 7 |
| G4 | Lenvatinib mesylate, 1 mg/kg, QD, p.o. + | 7 |
| | Antibody 1.14.4, 3 mg/kg, Q2D, i.p. | |

Note:
^{a}Dosing volume was 10 μL/g based on the body weight of the animal;
^{b}QD refers to once a day; Q2D refers to once every two days;
^{c}p.o. refers to orally; i.p. refers to intraperitoneal injection. The administration interval of the two drugs in the combination group was 0.5-1 h.

7.3. Dose Adjustment or Suspension

The animals were daily checked at the time of routine monitoring. If one or more of the following conditions occur, the administration should be suspended until the animal returns to be normal.

1) The animal's body weight loss is more than 20% of the initial body weight at the start of treatment (when the body weight loss is within 10% of the initial body weight at the start of treatment, the administration is continued).

2) After administration, the body temperature of the animal decreases and cannot be recovered.

3) After administration, the mobility of the animal is sluggish or abnormal.

During the experiment, no mice were subjected to suspension of administration due to weight loss.

7.4. Termination of the Experiment

The experiment was terminated one day after the final administration of Lenvatinib mesylate group.

7.5. Humane Endpoints for Experimental Animals

During the course of the experiment, animals should be euthanized if one or more of the following conditions occur.

1) When the tumor volume of the animal exceeds 3000 mm³, or the average tumor volume of the whole group exceeds 2000 mm³.

2) Tumor becomes anabrotic, necrotic or infected, and has not been recovered within one week.

3) The animal suffers from sluggish mobility, or paralysis.

4) The animal's body weight loss is more than 20% of the initial body weight at the start of treatment.

During the experiment, No. 52136 and No. 52158 mice in the G1 group were euthanized in advance because the tumor volume exceeded 3000 mm³. The body weight and tumor volume data of the two mice on the day of euthanasia were followed to the termination of the experiment.

7.6. Euthanasia

Animals were euthanized with excess CO₂ at the termination of the experiment or humane endpoints.

7.7. Experimental Indicators

7.7.1. Tumor Volume

Tumor volume was measured three times a week in two dimensions using a caliper, and the volume was expressed in mm³ using the formula: V=0.5 a×b² where a and b were the long and short diameters of the tumor, respectively.

7.7.2. Body Weight

Animals were weighed 2-3 times a week before tumor inoculation, during animal grouping and the treatment, and before the euthanasia.

7.7.3. General Clinical Observations

At the time of routine monitoring, the animals were checked daily for any effects of tumor growth, ulceration, mental state and behavior such as mobility, visual estimation of food and water consumption, body weight gain/loss, eye/hair matting and any other abnormal effect.

7.7.4. Tumor Weight and Photographing

At the termination of the experiment, the animals were photographed to record the tumor bearing state after euthanasia, and the tumors of the mice were weighed and photographed.

7.8. Evaluation Index of Drug Efficacy

7.8.1. Tumor Growth Inhibition (TGI_{TV}):

TGI_{TV}(%)=[1−(Tᵢ−T₀)/(Vᵢ−V₀)]×100%.

Tᵢ: tumor volume of the treatment group at day i following treatment; T₀: tumor volume of the treatment group at day 0 following treatment; Vᵢ: tumor volume of the control group at day i following treatment; V₀: tumor volume of the control group at day 0 following treatment.

7.8.2. Tumor Weight Inhibition (TGI_{TW}):

At the termination of the experiment, the animals were euthanized, the tumors were separated and weighed, and the difference of tumor weight between each group was calculated. Tumor weight inhibition (TGI_{TW}) was calculated by the formula:

TGI_{TW} (%)=(W_{C}−W_{T})/W_{T}×100%; where We means tumor weight of control group, W_{T} means tumor weight of treatment group.

7.9. Statistical Analysis

The original data measured and observed must be recorded. The original data was analyzed and the results were expressed by mean and standard error (Mean±SEM). The data of body weight, tumor volume and tumor weight was statistically analyzed using independent-samples t-test. All data were analyzed using SPSS, P<0.05 is considered to be statistically significant. The results of the analysis were considered both statistical significance and biological significance.

8. Results

8.1. Body Weight Changes

All the experimental animals were in a good state of mobility, diet, etc. During the treatment period, the body weight of the mice had a certain degree increase. There was no significant difference in body weight between the different treatment groups and hIgG4 control group (P>0.05). No. 52158 mouse in G1 group was euthanized on the 14th day after the start of treatment because the tumor volume exceeded 3000 mm³. The weight data of this mouse on the day of euthanasia was followed to the termination of the experiment and included in the statistical analysis. The body weight changes of all animals were shown in Table 7.

TABLE 7
Body weight changes

| | Animal | Days after the start of treatment/weight (g) | | | | | | | | | |
|---|---|---|---|---|---|---|---|---|---|---|---|
| Group | No. | 0 | 3 | 6 | 8 | 10 | 12 | 14 | 16 | 19 | 20 |
| G1 | 52138 | 19.7 | 19.2 | 19.6 | 19.6 | 20.2 | 20.8 | 22.3 | 22.4 | 21.2 | 22.1 |
| | 52115 | 19.7 | 19.0 | 20.0 | 19.9 | 20.7 | 19.3 | 21.6 | 22.6 | 21.6 | 21.6 |
| | 52175 | 19.7 | 18.9 | 19.7 | 20.3 | 20.7 | 18.9 | 20.3 | 20.5 | 22.0 | 22.6 |
| | 52105 | 20.0 | 19.6 | 20.2 | 20.1 | 20.5 | 20.9 | 22.0 | 21.7 | 21.2 | 21.9 |
| | 52136 | 20.3 | 19.0 | 20.1 | 19.7 | 20.5 | 20.2 | 22.6 | 23.3 | 23.3 | 23.3 |
| | 52142 | 21.6 | 21.0 | 22.5 | 22.3 | 23.0 | 23.7 | 24.7 | 25.2 | 24.3 | 22.5 |
| | 52158 | 20.2 | 19.4 | 19.8 | 21.0 | 21.6 | 22.8 | 24.1 | 24.1 | 24.1 | 24.1 |
| | Mean | 20.2 | 19.4 | 20.3 | 20.4 | 21.0 | 20.9 | 22.5 | 22.8 | 22.5 | 22.6 |
| | SD | 0.7 | 0.7 | 1.0 | 1.0 | 1.0 | 1.8 | 1.5 | 1.5 | 1.3 | 0.9 |
| | SEM | 0.3 | 0.3 | 0.4 | 0.4 | 0.4 | 0.7 | 0.6 | 0.6 | 0.5 | 0.3 |
| | CV | 3.4% | 3.8% | 5.0% | 4.7% | 4.6% | 8.4% | 6.6% | 6.8% | 6.0% | 3.8% |
| G2 | 52118 | 19.5 | 19.9 | 20.3 | 20.5 | 20.1 | 21.3 | 22.0 | 22.2 | 21.8 | 22.4 |
| | 52149 | 19.2 | 19.4 | 20.2 | 20.3 | 20.0 | 21.1 | 22.0 | 21.7 | 22.4 | 23.3 |
| | 52182 | 18.6 | 19.5 | 19.9 | 20.2 | 20.5 | 21.3 | 22.6 | 23.0 | 21.9 | 23.0 |
| | 52107 | 20.0 | 19.3 | 20.3 | 20.2 | 20.8 | 20.6 | 22.2 | 22.4 | 21.7 | 22.6 |
| | 52110 | 21.1 | 21.0 | 21.7 | 21.9 | 22.0 | 22.9 | 23.5 | 22.9 | 23.1 | 24.4 |
| | 52147 | 20.2 | 20.2 | 21.3 | 20.8 | 21.1 | 21.4 | 22.3 | 22.6 | 22.7 | 23.2 |
| | 52150 | 21.3 | 20.6 | 21.7 | 21.3 | 22.1 | 21.8 | 23.3 | 23.4 | 22.8 | 23.7 |
| | Mean | 20.0 | 20.0 | 20.8 | 20.7 | 20.9 | 21.5 | 22.6 | 22.6 | 22.3 | 23.2 |
| | SD | 1.0 | 0.6 | 0.8 | 0.6 | 0.8 | 0.7 | 0.6 | 0.6 | 0.6 | 0.7 |
| | SEM | 0.4 | 0.2 | 0.3 | 0.2 | 0.3 | 0.3 | 0.2 | 0.2 | 0.2 | 0.3 |
| | CV | 4.9% | 3.2% | 3.7% | 3.1% | 4.0% | 3.4% | 2.7% | 2.5% | 2.5% | 2.9% |
| G3 | 52104 | 18.6 | 18.8 | 19.4 | 19.5 | 20.2 | 20.1 | 20.9 | 21.6 | 21.2 | 21.5 |
| | 52116 | 19.5 | 19.2 | 20.1 | 20.6 | 21.1 | 21.1 | 22.9 | 22.8 | 23.0 | 23.2 |
| | 52169 | 19.4 | 18.9 | 19.8 | 20.4 | 20.1 | 21.1 | 22.0 | 22.6 | 23.0 | 23.2 |
| | 52123 | 20.0 | 19.7 | 20.6 | 20.7 | 21.5 | 21.4 | 22.2 | 22.5 | 21.9 | 22.6 |
| | 52143 | 20.2 | 19.3 | 20.3 | 20.3 | 20.4 | 20.7 | 22.0 | 21.9 | 21.4 | 22.1 |
| | 52153 | 20.8 | 20.3 | 21.2 | 21.0 | 21.7 | 22.2 | 23.0 | 23.0 | 22.6 | 23.2 |
| | 52156 | 20.8 | 20.2 | 20.8 | 21.0 | 21.4 | 21.6 | 23.0 | 22.8 | 20.3 | 22.9 |
| | Mean | 19.9 | 19.5 | 20.3 | 20.5 | 20.9 | 21.2 | 22.3 | 22.5 | 21.9 | 22.7 |
| | SD | 0.8 | 0.6 | 0.6 | 0.5 | 0.7 | 0.7 | 0.8 | 0.5 | 1.0 | 0.7 |
| | SEM | 0.3 | 0.2 | 0.2 | 0.2 | 0.3 | 0.3 | 0.3 | 0.2 | 0.4 | 0.2 |
| | CV | 4.0% | 3.1% | 3.0% | 2.5% | 3.2% | 3.2% | 3.4% | 2.3% | 4.6% | 2.9% |
| G4 | 52119 | 19.3 | 19.1 | 19.5 | 20.0 | 21.1 | 20.6 | 21.9 | 21.8 | 21.0 | 21.7 |
| | 52101 | 19.9 | 19.8 | 20.7 | 20.3 | 20.9 | 20.3 | 21.8 | 21.9 | 21.2 | 21.9 |
| | 52155 | 20.3 | 19.5 | 20.3 | 20.3 | 21.0 | 20.5 | 21.9 | 21.8 | 21.4 | 22.0 |
| | 52130 | 20.3 | 19.4 | 20.5 | 20.9 | 21.8 | 21.6 | 22.7 | 23.2 | 22.9 | 23.2 |
| | 52135 | 20.5 | 19.4 | 20.3 | 20.1 | 20.5 | 20.2 | 21.7 | 21.7 | 21.2 | 21.7 |
| | 52176 | 20.7 | 20.2 | 20.8 | 21.4 | 22.8 | 22.8 | 23.9 | 24.1 | 23.6 | 23.9 |
| | 52181 | 21.4 | 21.3 | 21.4 | 21.9 | 21.9 | 22.2 | 23.1 | 23.2 | 23.3 | 23.4 |
| | Mean | 20.3 | 19.8 | 20.5 | 20.7 | 21.4 | 21.2 | 22.4 | 22.5 | 22.1 | 22.5 |
| | SD | 0.7 | 0.7 | 0.6 | 0.7 | 0.8 | 1.0 | 0.8 | 1.0 | 1.1 | 0.9 |
| | SEM | 0.2 | 0.3 | 0.2 | 0.3 | 0.3 | 0.4 | 0.3 | 0.4 | 0.4 | 0.3 |
| | CV | 3.2% | 3.7% | 2.8% | 3.5% | 3.7% | 4.9% | 3.7% | 4.3% | 5.1% | 4.1% |

The body weight on the 16th day after the start of treatment was statistically analyzed using independent-samples t-test, and the result was shown in Table 8.

TABLE 8
Body weight on the 16th day

| | | | Weight (g)/Mean ± SEM | | | |
|---|---|---|---|---|---|---|
| Group | Treatment | Animals | Day 0 ^{a} | Day 16 ^{a} | P^{b} | Changes |
| G1 | hIgG4 3 mg/kg | 7 | 20.2 ± 0.3 | 22.8 ± 0.6 | — | +2.6 |
| G2 | Antibody 1.14.4 3 mg/kg | 7 | 20.0 ± 0.4 | 22.6 ± 0.2 | 0.720 | +2.6 |
| G3 | Lenvatinib mesylate 1 mg/kg | 7 | 19.9 ± 0.3 | 22.5 ± 0.2 | 0.565 | +2.6 |
| G4 | Lenvatinib mesylate 1 mg/kg + | 7 | 20.3 ± 0.2 | 22.5 ± 0.4 | 0.671 | +2.2 |
| | Antibody 1.14.4 3 mg/kg | | | | | |

Note:
^{a} Day 0 is the day of the start of treatment (i.e., the day of animal grouping), Day 16 is the 16th day after the start of treatment;
^{b}p value vs control group.

8.2. Tumor Volume Results

Tumor growth of all animals was closely monitored during the whole period of the experiment. The tumor volume of all animals was measured three times a week and the results were recorded. The tumor volume data was shown in Table 9. No. 52158 mouse in G group was euthanized on the 14th day after the start of treatment because the tumor volume exceeded 3000 mm³. The tumor growth data of this mouse on the day of euthanasia was followed to the termination of the experiment and included in the statistical analysis.

TABLE 9
Tumor volume

| | Animal | Days after the start of treatment/tumor volume (mm³) | | | | | | | | | |
|---|---|---|---|---|---|---|---|---|---|---|---|
| Group | No. | 0 | 3 | 6 | 8 | 10 | 12 | 14 | 16 | 19 | 20 |
| G1 | 52138 | 98.6 | 275.2 | 473.6 | 659.1 | 869.1 | 924.8 | 930.3 | 1265.3 | 1406.0 | 1607.2 |
| | 52115 | 108.8 | 306.8 | 836.2 | 1229.0 | 1128.4 | 1297.1 | 1584.6 | 1882.1 | 1737.5 | 1859.5 |
| | 52175 | 95.6 | 222.6 | 453.6 | 672.1 | 835.5 | 1033.7 | 1417.4 | 1606.0 | 1902.4 | 2305.1 |
| | 52105 | 82.9 | 286.1 | 602.2 | 712.2 | 999.8 | 1262.4 | 1260.6 | 1551.3 | 2173.4 | 3175.2 |
| | 52136 | 112.9 | 474.6 | 947.1 | 1198.3 | 1575.3 | 2001.3 | 2514.7 | 3080.0 | 3080.0 | 3080.0 |
| | 52142 | 121.5 | 378.7 | 979.2 | 868.6 | 1125.2 | 1659.1 | 1316.0 | 1783.5 | 1757.9 | 1951.0 |
| | 52158 | 98.9 | 527.6 | 1138.6 | 1934.9 | 2415.4 | 2484.3 | 3618.4 | 3618.4 | 3618.4 | 3618.4 |
| | Mean | 103 | 353 | 776 | 1039 | 1278 | 1523 | 1806 | 2112 | 2239 | 2514 |
| | SD | 13 | 112 | 268 | 462 | 558 | 561 | 939 | 881 | 807 | 773 |
| | SEM | 5 | 42 | 101 | 175 | 211 | 212 | 355 | 333 | 305 | 292 |
| | CV | 12.4% | 31.8% | 34.6% | 44.4% | 43.6% | 36.8% | 52.0% | 41.7% | 36.0% | 30.8% |
| G2 | 52118 | 118.2 | 177.1 | 288.1 | 323.2 | 335.8 | 296.5 | 253.7 | 287.9 | 306.0 | 399.4 |
| | 52149 | 86.2 | 134.6 | 308.7 | 433.2 | 457.8 | 693.6 | 710.0 | 994.6 | 1173.0 | 1267.0 |
| | 52182 | 110.7 | 305.3 | 739.6 | 887.1 | 1046.6 | 1557.3 | 1756.4 | 2427.2 | 3093.0 | 3146.2 |
| | 52107 | 75.5 | 176.8 | 294.8 | 405.3 | 499.7 | 887.5 | 1053.1 | 1245.4 | 1976.9 | 1777.2 |
| | 52110 | 107.4 | 279.0 | 361.3 | 387.6 | 507.7 | 810.9 | 1049.4 | 980.7 | 1194.0 | 982.9 |
| | 52147 | 99.2 | 210.5 | 318.8 | 391.6 | 638.6 | 898.6 | 1010.9 | 1192.0 | 2068.6 | 2071.5 |
| | 52150 | 121.1 | 272.7 | 617.4 | 726.5 | 765.6 | 1036.5 | 1245.6 | 1572.2 | 1642.7 | 1783.3 |
| | Mean | 103 | 222 | 418 | 508 | 607 | 883 | 1011 | 1243 | 1636 | 1632 |
| | SD | 17 | 64 | 183 | 212 | 237 | 379 | 462 | 653 | 876 | 876 |
| | SEM | 6 | 24 | 69 | 80 | 90 | 143 | 174 | 247 | 331 | 331 |
| | CV | 16.4% | 28.8% | 43.7% | 41.8% | 39.0% | 43.0% | 45.6% | 52.5% | 53.5% | 53.7% |
| | TGI | — | 0.522 | 0.531 | 0.567 | 0.571 | 0.451 | 0.466 | 0.433 | 0.282 | 0.365 |
| G3 | 52104 | 87.7 | 195.2 | 432.7 | 620.5 | 964.3 | 1093.4 | 1170.2 | 1341.3 | 1427.3 | 1488.5 |
| | 52116 | 102.8 | 340.1 | 579.3 | 886.4 | 1269.5 | 1248.3 | 1678.0 | 2139.3 | 2792.6 | 2915.3 |
| | 52169 | 114.6 | 329.8 | 592.7 | 718.2 | 974.5 | 1425.3 | 1754.2 | 2366.9 | 2701.0 | 2973.9 |
| | 52123 | 136.5 | 249.5 | 538.7 | 604.1 | 630.4 | 613.4 | 614.3 | 892.3 | 994.4 | 936.1 |
| | 52143 | 72.8 | 232.6 | 370.4 | 520.3 | 481.7 | 538.7 | 430.5 | 437.1 | 503.7 | 582.0 |
| | 52153 | 96.2 | 264.5 | 456.2 | 498.9 | 777.4 | 807.9 | 1120.2 | 1179.2 | 2127.4 | 2258.9 |
| | 52156 | 108.1 | 269.1 | 468.5 | 605.0 | 612.2 | 869.2 | 829.2 | 971.9 | 821.4 | 925.6 |
| | Mean | 103 | 269 | 491 | 636 | 816 | 942 | 1085 | 1333 | 1624 | 1726 |
| | SD | 20 | 52 | 82 | 132 | 271 | 328 | 504 | 692 | 923 | 990 |
| | SEM | 8 | 19 | 31 | 50 | 103 | 124 | 190 | 2.61 | 349 | 374 |
| | CV | 19.8% | 19.2% | 16.6% | 20.7% | 33.3% | 34.8% | 46.4% | 51.9% | 56.8% | 57.4% |
| | TGI | — | 0.337 | 0.423 | 0.43 | 0.393 | 0.4 | 0.423 | 0.388 | 0.288 | 0.327 |
| G4 | 52119 | 88.4 | 167.6 | 283.1 | 274.9 | 326.0 | 426.2 | 424.3 | 549.2 | 877.4 | 750.7 |
| | 52101 | 109.0 | 182.9 | 412.8 | 460.7 | 475.7 | 540.2 | 569.9 | 627.3 | 698.5 | 756.6 |
| | 52155 | 99.7 | 345.3 | 722.7 | 683.5 | 927.3 | 821.5 | 835.8 | 736.1 | 1012.4 | 1107.5 |
| | 52130 | 121.0 | 304.0 | 634.0 | 622.3 | 836.1 | 967.5 | 1045.8 | 1137.2 | 1643.5 | 1890.7 |
| | 52135 | 82.7 | 187.5 | 238.4 | 254.8 | 358.0 | 360.3 | 394.0 | 417.5 | 508.1 | 555.8 |
| | 52176 | 119.5 | 318.6 | 603.9 | 660.1 | 909.4 | 1169.1 | 1095.3 | 1323.2 | 1450.1 | 1473.1 |
| | 52181 | 97.8 | 262.3 | 350.3 | 328.7 | 492.5 | 549.6 | 537.9 | 562.6 | 718.9 | 696.6 |
| | Mean | 103.0 | 253.0 | 464.0 | 469.0 | 618.0 | 691.0 | 700.0 | 765.0 | 987.0 | 1033.0 |
| | SD | 15 | 73 | 189 | 187 | 264 | 301 | 291 | 336 | 417 | 488 |
| | SEM | 6 | 28 | 71 | 71 | 100 | 114 | 110 | 127 | 158 | 185 |
| | CV | 14.3% | 28.9% | 40.8% | 39.8% | 42.7% | 43.6% | 41.5% | 44.0% | 42.2% | 47.3% |
| | TGI | — | 0.401 | 0.464 | 0.608 | 0.562 | 0.586 | 0.649 | 0.671 | 0.586 | 0.614 |

On the 16th day after the start of treatment, the tumor volume on the 16th day after the start of treatment was statistically analyzed using independent-samples t-test, and the result was shown in Table 10. It can be seen that the combination of Lenvatinib mesylate (1 mg/kg) and antibody 1.14.4 (3 mg/kg) exhibits a more significant antitumor activity than the monotherapy group.

TABLE 10
Tumor volume on the 16th day

| | | | Tumor volume | | | |
|---|---|---|---|---|---|---|
| | | | (mm³)/Mean ± SEM | | | |
| Group | Treatment | Animals | Day 0 ^{a} | Day 16 ^{a} | TGI_{TV} (%) | P^{b} |
| G1 | hIgG4 3 mg/kg | 7 | 103 ± 5 | 2112 ± 333 | — | — |
| G2 | antibody 1.14.4 3 mg/kg | 7 | 103 ± 6 | 1243 ± 247 | 43.3 | 0.058 |
| G3 | Lenvatinib mesylate 1 mg/kg | 7 | 103 ± 8 | 1333 ± 261 | 38.8 | 0.090 |
| G4 | Lenvatinib mesylate 1 mg/kg + | 7 | 103 ± 6 | 765 ± 127 | 67.1 | 0.006 |
| | antibody 1.14.4 3 mg/kg | | | | | |

Note:
^{a} Day 0 is the day of the start of treatment (i.e., the day of animal grouping), Day 16 is the 16th day after the start of treatment;
^{b}p value vs control group (G1).

8.3. Synergy Analysis

Synergy score was calculated using the following formula described in Clarke R. Issues in experimental design and endpoint analysis in the study of experimental cytotoxic agents in vivo in breast cancer and other models[J]. Breast Cancer Research & Treatment, 1997, 46(2-3):255-278, which is incorporated by reference in its entirety:

Synergy score=((A/C)×(B/C))/(AB/C);

where A is RTV value of drug A; B is RTV value of drug B; C is RTV value of vehicle control; AB is RTV value of combination treatment with A and B respectively.
Synergy score>1 represents synergistic;
Synergy score=1 represents additive;
Synergy score<1 represents antagonistic.

Relative tumor volume (RTV) was calculated using the following formula:

RTV=Vₜ/V₀

where V₀ and Vₜ are the average tumor volume at the start of treatment (day 0) and the average tumor volume on a certain time point (day t).

The results of average tumor volume on the 20th day and synergy score were shown in Table 11.

TABLE 11
Synergy analysis

| | | Tumor volume | | Synergy |
|---|---|---|---|---|
| | | (mm³)/Mean ± SEM | | score |
| Group | Treatment | Day 0 | Day 20 | @ D20 |
| G1 | hIgG4 3 mg/kg | 103 ± 5 | 2514 ± 292 | — |
| G2 | Antibody 1.14.4 3 mg/kg | 103 ± 6 | 1632 ± 331 | — |
| G3 | Lenvatinib mesylate | 103 ± 8 | 1726 ± 374 | — |
| | 1 mg/kg | | | |
| G4 | Lenvatinib mesylate | 103 ± 6 | 1033 ± 185 | 1.08 |
| | 1 mg/kg + Antibody | | | |
| | 1.14.4 3 mg/kg | | | |

Synergistic effect between Lenvatinib mesylate and antibody 1.14.4 was demonstrated as synergy score was larger than 1 in the combination group G4.

8.4. Tumor Weight Results

In this experiment, all animals were euthanized on the 20th day after the start of treatment, and tumors were separated, weighed and photographed. The individual tumor weight data was shown in Table 12.

TABLE 12
Tumor weight

| | Animal | Tumor weight |
|---|---|---|
| Group | No. | (g) |
| G1 | 52138 | 1.021 |
| | 52115 | 1.168 |
| | 52175 | 1.583 |
| | 52105 | 1.353 |
| | 52136 | 2.074 |
| | 52142 | 1.509 |
| | 52158 | 2.365 |
| | Mean | 1.582 |
| | SD | 0.483 |
| | SEM | 0.183 |
| | CV | 30.5% |
| G2 | 52118 | 0.329 |
| | 52149 | 0.882 |
| | 52182 | 2.554 |
| | 52107 | 1.823 |
| | 52110 | 1.200 |
| | 52147 | 1.337 |
| | 52150 | 1.093 |
| | Mean | 1.333 |
| | SD | 0.741 |
| | SEM | 0.280 |
| | CV | 55.6% |
| | TGI_{TW} | 15.8% |
| G3 | 52104 | 1.039 |
| | 52116 | 2.048 |
| | 52169 | 2.485 |
| | 52123 | 0.652 |
| | 52143 | 0.408 |
| | 52153 | 1.362 |
| | 52156 | 0.715 |
| | Mean | 1.244 |
| | SD | 0.772 |
| | SEM | 0.292 |
| | CV | 62.0% |
| | TGI_{TW} | 21.3% |
| G4 | 52119 | 0.818 |
| | 52101 | 0.641 |
| | 52155 | 0.716 |
| | 52130 | 1.545 |
| | 52135 | 0.457 |
| | 52176 | 1.198 |
| | 52181 | 0.536 |
| | Mean | 0.844 |
| | SD | 0.391 |
| | SEM | 0.148 |
| | CV | 46.4% |
| | TGI_{TW} | 46.6% |

The tumor weight was statistically analyzed using independent-samples t-test, and the result was shown in Table 13. It can be seen that the combination of Lenvatinib mesylate (1 mg/kg) and antibody 1.14.4 (3 mg/kg) exhibits a more significant antitumor activity than the monotherapy group.

TABLE 13
Tumor weight statistical analysis

| | | | Tumor weight (g) | TGI_{TV} | |
|---|---|---|---|---|---|
| Group | Treatment | Animals | Mean ± SEM | (%) | P^{a} |
| G1 | hIgG4 3 mg/kg | 7 | 1.582 ± 0.183 | — | — |
| G2 | antibody 1.14.4 | 7 | 1.333 ± 0.280 | 15.8 | 0.470 |
| | 3 mg/kg | | | | |
| G3 | Lenvatinib mesylate | 7 | 1.244 ± 0.292 | 21.3 | 0.346 |
| | 1 mg/kg | | | | |
| G4 | Lenvatinib mesylate | 7 | 0.844 ± 0.148 | 65.8 | 0.009 |
| | 1 mg/kg + antibody | | | | |
| | 1.14.4 3 mg/kg | | | | |

Note:
^{a}p value vs control group (G1).

### Embodiment 2: Pharmacodynamic Study of Antibody 2E5 Monotherapy or in Combination with Lenvatinib Mesylate on B-hPD-1 Humanized Mouse MC38-hPD-L1 Colon Cancer Animal Model

1. Objective of the Study

The objective of the study was to evaluate the therapeutic efficacy of antibody 2E5 as a single agent or in combination with Lenvatinib mesylate in the treatment of subcutaneous MC38-hPD-L1 transplanted tumor.

2. Reagents and Equipment

TABLE 14
Reagents and equipment

| Reagents | | | | | |
|---|---|---|---|---|---|
| or | | | Storage | | Physical |
| equipment | Supplier | Art. No. | condition | Lot. No. | form |
| DMEM | Cellgro | 10-013-CVR | 4° C. | — | — |
| FBS | HyClone | SH30406.02 | −80° C. | — | — |
| PBS | Gibco | 20012-027 | 4° C. | — | — |
| Vernier | Hexagon | 00534220 | — | — | — |
| caliper | Metrology | | | | |
| 0.9% | — | — | — | — | — |
| Sodium | | | | | |
| chloride | | | | | |
| injection | | | | | |
| hIgG4 | CROWN | C0004-4 | — | AB170091 | 5.1 mg/mL |
| | BIO- | | | | solution in |
| | SCIENCE | | | | physio- |
| | INC | | | | logical |
| | TAICANG | | | | saline |
| Lenvatinib | Eisai Co., | — | — | 164H0501 | powder |
| mesylate | Ltd. | | | | |

3. Test Sample

Antibody 2E5 was prepared according to WO2018053709A1 (Examples 1-3).

4. Formulation

4.1. Formulation of hIgG4

5.1 mg/mL solution of hIgG4 in physiological saline was diluted with 0.9% sodium chloride injection to a concentration of 0.2 mg/mL for use. The prepared solution was stored at 2-8° C. when not in use, and allowed to stand to warm to room temperature before use.

4.2. Formulation of Different Concentration of Lenvatinib Mesylate

Lenvatinib mesylate powder was dissolved in 3 mmol/L HCl solution to prepare 3 mg/mL stock solution of Lenvatinib mesylate. The stock solution was stored at 4° C. when not in use.

1 mg/mL Lenvatinib mesylate solution was prepared by diluting 0.12 mL of 3 mg/mL stock solution with 0.24 mL of 3 mmol/L HCl solution. 0.36 mL of 1 mg/mL Lenvatinib mesylate solution was further diluted with 3.24 mL of 3 mmol/L HCl solution to prepare 0.1 mg/mL Lenvatinib mesylate solution for use. The prepared solution was stored at 2-8° C. when not in use, and allowed to stand to warm to room temperature before use.

4.3. Formulation of Antibody 2E5

Antibody 2E5 was diluted with 0.9% sodium chloride injection to prepare 0.1 mg/mL and 0.2 mg/mL antibody 2E5 solution for use. The prepared solution was stored at 2-8° C. when not in use, and allowed to stand to warm to room temperature before use.

5. Animals

Species: Mus musculus;

Strain: C57BL/6;

Name: B-hPD-1 humanized mice;

Sex: male;

Body weight: 20-28 g

Age: 7 weeks;

Number of animals: 100 (60 plus 40 spare) mice;

Animal supplier: Biocytogen Jiangsu Co., Ltd;

License number: SCXK (Jiangsu) 2016-0004;

Animal Certificate No.: 201803133.

6. Animal Housing and Care

Animals were housed in the SPF animal laboratory of Experiment Animal Center in Biocytogen Beijing Co., Ltd using individually ventilated cages (IVC). Animals were acclimated to the environment for 3-7 days prior to initiate the study.

Temperature: 20-26° C.;

Humidity: 40%-70%;

Light: 12 hours on and 12 hours off;

Cage: Cage was made of PEI with a volume of 320 mm×200 mm×135 mm. Corn cobs were used for animal bedding, which were sterilized by autoclave before use. Beddings were changed once a week. Each cage was identified by a cage card marking the number of animals, gender, strain, receiving date, group number, and starting date of the experiment.

Diet and water: Reverse osmosis (RO) water was autoclaved before use. Animals had free access to SPF mouse feed and sterile drinking water.

Animal identification: Each mice was identified by ear tag.

7. Experimental Procedure

7.1. Cell Culture

Mouse colon cancer cells MC38 were purchased from Shun Ran (Shanghai) Biotechnology Co., Ltd., and the cells were cultured in a 37° C., 5% CO₂ incubator supplemented with Dulbecco's Modified Eagle's medium containing 10% inactivated fetal bovine serum. MC38 cells were genetically modified to overexpress human PD-L1 and knock out murine PD-L1, the modified cells were named as MC38-hPD-L1 cells.

7.2. Tumor Inoculation, Group and Dosage Regimen

MC38-hPD-L1 tumor cells were resuspended in 1×PBS to a concentration of 5×10⁵ cells per 0.1 mL. Each of the B-hPD-1 humanized mice was subcutaneously inoculated at the right lateral thorax region with 0.1 mL of the cell suspension. When the average tumor volume reached about 77 mm³, the mice with moderate tumor volume were selected and randomly divided into 6 groups according to tumor volume, with 10 mice in each group, and the treatment was initiated on the same day. The dosage regimen was shown in Table 15.

TABLE 15
Dosage regimen

| | Treatment^{a} | | Animals |
|---|---|---|---|
| Group | Dose^{b}, route | Frequency^{c} | No. |
| G1 | hIgG4, 2 mg/kg, i.p. | 0, 4, 8, 11, 13, 16; | 10 |
| | | a total of 6 times | |
| G2 | Antibody 2E5, | 0, 4, 8, 11, 13, 16; | 10 |
| | 1 mg/kg, i.p. | a total of 6 times | |
| G3 | Antibody 2E5, | 0, 4, 8, 11, 13, 16; | 10 |
| | 2 mg/kg, i.p. | a total of 6 times | |
| G4 | Lenvatinib mesylate, | QD; a total of 18 times | 10 |
| | 1 mg/kg, p.o. | | |
| G5 | Lenvatinib mesylate, | Lenvatinib mesylate: | 10 |
| | 1 mg/kg, p.o. + | QD; a total of 18 times | |
| | Antibody 2E5, | Antibody 2E5: 0, 4, 8, 11, | |
| | 1 mg/kg, i.p. | 13, 16; a total of 6 times | |
| G6 | Lenvatinib mesylate, | Lenvatinib mesylate: | 10 |
| | 1 mg/kg, p.o. + | QD; a total of 18 times | |
| | Antibody 2E5, | Antibody 2E5: 0, 4, 8, 11, | |
| | 2 mg/kg, i.p. | 13, 16; a total of 6 times | |

Note:
^{a}The administration interval of the two drugs in the combination group (i.e., G5 and G6) was 0.5-1 h;
^{b}Dosing volume was 10 μL/g based on the body weight of the animal;
^{c}p.o. means orally; i.p. means intraperitoneal injection; QD means once a day; 0, 4, 8, 11, 13, 16 means days after animal grouping (i.e., the day of the start of treatment).

7.3. Dose Adjustment or Suspension

The animals were daily checked at the time of routine monitoring. If one or more of the following conditions occur, the administration should be suspended until the animal returns to be normal:

1) The animal's body weight loss is more than 20% of the initial body weight at the start of treatment (when the body weight loss is within 10% of the initial body weight at the start of treatment, the administration is continued).

2) After administration, the body temperature of the animal decreases and cannot be recovered.

3) After administration, the mobility of the animal is sluggish or abnormal.

During the experiment, no mice were subjected to suspension of administration due to the above reasons.

7.4. Termination of the Experiment

The experiment was terminated 2 hours after the final administration of Lenvatinib mesylate on the 17^{th} day after the start of treatment.

7.5. Humane Endpoints for Experimental Animals

During the course of the experiment, animals should be euthanized if one or more of the following conditions occur:

1) When the tumor volume of the animal exceeds 3000 mm³, or the average tumor volume of the whole group exceeds 2000 mm³.

2) Tumor becomes anabrotic, necrotic or infected, and has not been recovered within one week.

3) The animal suffers from sluggish mobility, or paralysis.

4) The animal's body weight loss is more than 20% of the initial body weight at the start of treatment.

During the experiment, No. 55191 mouse in the G1 group was euthanized in advance because the tumor volume exceeded 3000 mm³ on the 16^{th} day after the start of treatment. No mice was euthanized in advance due to other clinical symptoms.

7.6. Euthanasia

Animals were euthanized with excess CO₂ at the termination of the experiment or humane endpoints.

7.7. Experimental Indicators

7.7.1. Tumor Volume:

Tumor volume was measured 2-3 times a week in two dimensions using a caliper, and the volume was expressed in mm³ using the formula: V=0.5 a×b² where a and b were the long and short diameters of the tumor, respectively.

7.7.2. Body Weight

Animals were weighed 2-3 times a week before tumor inoculation, during animal grouping and the treatment, and before the euthanasia.

7.7.3. General Clinical Observations

At the time of routine monitoring, the animals were checked daily for any effects of tumor growth, ulceration, mental state and behavior such as mobility, visual estimation of food and water consumption, body weight gain/loss, eye/hair matting and any other abnormal effect.

7.7.4. Tumor Weight and Photographing

At the termination of the experiment, the animals were photographed to record the tumor bearing state after euthanasia, and the tumors of the mice were weighed and photographed.

7.8. Evaluation Index of Drug Efficacy

7.8.1. Tumor Growth Inhibition (TGI_{TV}):

TGI_{TV}(%)=[1−(Tᵢ−T₀)/(Vᵢ−V₀)]×100%.

Tᵢ: tumor volume of the treatment group at day i following treatment; T₀: tumor volume of the treatment group at day 0 following treatment; Vᵢ: tumor volume of the control group at day i following treatment; V₀: tumor volume of the control group at day 0 following treatment.

7.8.2. Tumor Weight Inhibition (TGI_{TW}):

At the termination of the experiment, the animals were euthanized, the tumors were separated and weighed, and the difference of tumor weight between each group was calculated. Tumor weight inhibition (TGI_{TW}) was calculated by the formula:

TGI_{TW} (%)=(W_{C}−W_{T})/W_{T}×100%; where We means tumor weight of control group, W_{T} means tumor weight of treatment group.

7.9. Statistical Analysis

The original data measured and observed must be recorded. The original data was analyzed and the results were expressed by mean and standard error (Mean±SEM). The data of body weight, tumor volume and tumor weight was statistically analyzed using independent-samples t-test. All data were analyzed using SPSS, P<0.05 is considered to be statistically significant.

8. Results

8.1. Body Weight Changes

All the experimental animals were in a good state of mobility, diet, etc. During the treatment period, the body weight of the mice had a certain degree increase. No. 55191 mouse in the G1 group was euthanized in advance because the tumor volume exceeded 3000 mm³ on the 16^{th} day after the start of treatment. The weight data of this mouse on the day of euthanasia was followed to the termination of the experiment and included in the statistical analysis. The body weight changes of all animals were shown in Table 16.

TABLE 16
Body weight change

| | Animal | Days after the start of treatment/body weight (g) | | | | | | |
|---|---|---|---|---|---|---|---|---|
| Group | No. | 0 | 4 | 8 | 10 | 12 | 15 | 17 |
| G1 | 55106 | 24.4 | 23.9 | 25.7 | 25.6 | 27.8 | 28.0 | 28.3 |
| | 55115 | 24.8 | 24.9 | 26.5 | 26.3 | 28.2 | 28.8 | 30.2 |
| | 55136 | 23.4 | 23.8 | 25.7 | 25.2 | 26.6 | 27.6 | 27.9 |
| | 55151 | 27.2 | 27.0 | 28.4 | 28.3 | 29.8 | 30.9 | 31.7 |
| | 55156 | 24.7 | 23.5 | 25.8 | 25.8 | 27.6 | 28.0 | 30.2 |
| | 55157 | 26.4 | 26.5 | 28.4 | 28.2 | 29.7 | 29.8 | 31.1 |
| | 55164 | 24.5 | 24.4 | 26.3 | 25.8 | 27.0 | 27.8 | 28.0 |
| | 55188 | 26.0 | 25.7 | 27.2 | 26.6 | 27.9 | 29.1 | 29.7 |
| | 55191 | 23.9 | 24.1 | 26.0 | 25.6 | 26.7 | 27.3 | 27.3 |
| | 55194 | 23.9 | 23.6 | 24.7 | 24.7 | 25.6 | 25.9 | 26.0 |
| | Mean | 24.9 | 24.7 | 26.5 | 26.2 | 27.7 | 28.3 | 29.0 |
| | SD | 1.2 | 1.3 | 1.2 | 1.2 | 1.3 | 1.4 | 1.8 |
| | SEM | 0.4 | 0.4 | 0.4 | 0.4 | 0.4 | 0.4 | 0.6 |
| | CV | 4.9% | 5.1% | 4.5% | 4.6% | 4.8% | 4.9% | 6.2% |
| G2 | 55122 | 27.3 | 27.2 | 28.6 | 27.5 | 28.8 | 30.0 | 29.6 |
| | 55131 | 27.6 | 27.7 | 29.5 | 29.0 | 30.0 | 31.2 | 31.8 |
| | 55147 | 25.4 | 21.8 | 26.9 | 26.7 | 27.9 | 28.7 | 29.2 |
| | 55150 | 23.8 | 23.3 | 25.0 | 24.3 | 25.5 | 26.9 | 27.6 |
| | 55161 | 22.1 | 25.0 | 23.2 | 22.9 | 23.2 | 23.8 | 24.2 |
| | 55168 | 24.5 | 25.2 | 26.7 | 26.7 | 27.5 | 28.9 | 29.5 |
| | 55170 | 21.9 | 23.6 | 24.3 | 24.5 | 26.0 | 26.4 | 26.9 |
| | 55178 | 23.9 | 25.1 | 27.0 | 26.4 | 27.8 | 28.4 | 28.2 |
| | 55184 | 24.9 | 25.2 | 27.5 | 26.7 | 28.0 | 29.1 | 29.5 |
| | 55190 | 27.3 | 27.0 | 28.8 | 27.4 | 28.9 | 29.4 | 29.8 |
| | Mean | 24.9 | 25.1 | 26.8 | 26.2 | 27.4 | 28.3 | 28.6 |
| | SD | 2.1 | 1.9 | 2.0 | 1.8 | 2.0 | 2.1 | 2.1 |
| | SEM | 0.7 | 0.6 | 0.6 | 0.6 | 0.6 | 0.7 | 0.7 |
| | CV | 8.3% | 7.4% | 7.6% | 6.9% | 7.2% | 7.4% | 7.2% |
| G3 | 55108 | 21.9 | 21.4 | 22.7 | 21.8 | 22.5 | 23.9 | 24.2 |
| | 55125 | 23.5 | 22.9 | 25.0 | 24.7 | 25.2 | 26.5 | 27.1 |
| | 55135 | 28.2 | 27.9 | 28.7 | 27.7 | 28.5 | 30.5 | 30.8 |
| | 55146 | 23.3 | 23.7 | 24.7 | 23.8 | 24.4 | 25.8 | 26.0 |
| | 55149 | 24.1 | 24.0 | 26.1 | 25.1 | 25.8 | 27.8 | 27.8 |
| | 55167 | 25.9 | 26.6 | 28.1 | 26.4 | 27.1 | 28.9 | 30.0 |
| | 55171 | 26.3 | 27.3 | 28.4 | 26.9 | 26.0 | 26.7 | 27.6 |
| | 55173 | 26.2 | 25.9 | 28.2 | 26.8 | 27.6 | 28.6 | 29.3 |
| | 55182 | 23.7 | 23.2 | 24.8 | 23.4 | 24.4 | 25.6 | 25.5 |
| | 55186 | 26.6 | 26.3 | 27.5 | 27.6 | 28.1 | 29.5 | 30.0 |
| | Mean | 25.0 | 24.9 | 26.4 | 25.4 | 26.0 | 27.4 | 27.8 |
| | SD | 1.9 | 2.2 | 2.0 | 2.0 | 1.9 | 2.0 | 2.2 |
| | SEM | 0.6 | 0.7 | 0.6 | 0.6 | 0.6 | 0.6 | 0.7 |
| | CV | 7.8% | 8.7% | 7.8% | 7.8% | 7.3% | 7.4% | 7.8% |
| G4 | 55103 | 25.1 | 25.8 | 26.5 | 26.2 | 27.2 | 28.0 | 28.7 |
| | 55104 | 28.0 | 27.7 | 29.5 | 29.7 | 29.7 | 30.6 | 31.8 |
| | 55123 | 26.0 | 26.4 | 27.5 | 27.0 | 27.5 | 28.2 | 29.5 |
| | 55139 | 25.5 | 25.2 | 25.9 | 25.3 | 26.3 | 27.3 | 27.9 |
| | 55155 | 27.4 | 27.0 | 27.3 | 27.2 | 27.3 | 28.0 | 29.2 |
| | 55163 | 22.7 | 22.6 | 23.8 | 23.7 | 23.9 | 25.5 | 26.1 |
| | 55165 | 19.6 | 19.6 | 20.9 | 20.2 | 20.6 | 21.9 | 22.3 |
| | 55175 | 23.0 | 23.1 | 25.5 | 24.5 | 25.3 | 26.8 | 27.0 |
| | 55187 | 25.2 | 25.3 | 26.7 | 26.1 | 26.7 | 27.4 | 28.0 |
| | 55198 | 26.0 | 25.8 | 26.4 | 26.8 | 27.5 | 29.2 | 29.7 |
| | Mean | 24.9 | 24.9 | 26.0 | 25.7 | 26.2 | 27.3 | 28.0 |
| | SD | 2.5 | 2.4 | 2.3 | 2.5 | 2.5 | 2.3 | 2.6 |
| | SEM | 0.8 | 0.8 | 0.7 | 0.8 | 0.8 | 0.7 | 0.8 |
| | CV | 10.0% | 9.8% | 8.9% | 9.8% | 9.5% | 8.5% | 9.1% |
| G5 | 55105 | 23.9 | 23.8 | 24.5 | 24.0 | 23.8 | 24.8 | 24.2 |
| | 55112 | 27.3 | 26.8 | 27.9 | 27.1 | 27.0 | 28.6 | 29.0 |
| | 55120 | 28.5 | 28.5 | 29.2 | 28.2 | 29.3 | 30.0 | 29.6 |
| | 55140 | 23.8 | 23.9 | 24.9 | 24.3 | 24.2 | 25.4 | 25.0 |
| | 55143 | 26.3 | 25.7 | 26.5 | 25.7 | 26.0 | 26.7 | 26.7 |
| | 55154 | 21.9 | 21.8 | 22.3 | 22.9 | 23.2 | 24.6 | 25.5 |
| | 55162 | 23.3 | 24.2 | 25.0 | 25.4 | 25.7 | 26.2 | 27.4 |
| | 55180 | 24.9 | 25.3 | 25.0 | 25.4 | 24.9 | 26.5 | 27.2 |
| | 55192 | 24.3 | 23.8 | 24.1 | 23.5 | 24.0 | 24.2 | 24.5 |
| | 55193 | 25.2 | 25.2 | 26.7 | 26.5 | 26.9 | 27.8 | 28.2 |
| | Mean | 24.9 | 24.9 | 25.6 | 25.3 | 25.5 | 26.5 | 26.7 |
| | SD | 2.0 | 1.9 | 2.0 | 1.7 | 1.9 | 1.9 | 1.9 |
| | SEM | 0.6 | 0.6 | 0.6 | 0.5 | 0.6 | 0.6 | 0.6 |
| | CV | 7.9% | 7.5% | 7.8% | 6.6% | 7.3% | 7.1% | 7.1% |
| G6 | 55101 | 25.0 | 24.7 | 25.3 | 24.9 | 24.7 | 25.1 | 25.8 |
| | 55109 | 22.6 | 22.8 | 23.8 | 23.4 | 22.4 | 24.2 | 24.4 |
| | 55113 | 23.3 | 24.0 | 24.2 | 24.0 | 23.9 | 24.8 | 24.8 |
| | 55121 | 23.6 | 23.5 | 25.0 | 24.1 | 24.1 | 24.6 | 25.2 |
| | 55127 | 25.2 | 25.6 | 26.0 | 25.7 | 25.3 | 26.6 | 27.0 |
| | 55159 | 27.2 | 26.9 | 28.0 | 27.5 | 27.8 | 29.3 | 29.4 |
| | 55160 | 25.6 | 25.7 | 27.2 | 26.7 | 27.0 | 27.8 | 28.2 |
| | 55185 | 27.4 | 27.6 | 28.2 | 28.0 | 28.6 | 29.9 | 29.9 |
| | 55189 | 23.0 | 23.1 | 23.3 | 23.5 | 23.2 | 23.6 | 24.2 |
| | 55199 | 26.7 | 25.5 | 26.5 | 26.2 | 26.7 | 27.7 | 28.3 |
| | Mean | 25.0 | 24.9 | 25.8 | 25.4 | 25.4 | 26.4 | 26.7 |
| | SD | 1.8 | 1.6 | 1.7 | 1.7 | 2.1 | 2.2 | 2.1 |
| | SEM | 0.6 | 0.5 | 0.5 | 0.5 | 0.7 | 0.7 | 0.7 |
| | CV | 7.1% | 6.4% | 6.7% | 6.6% | 8.2% | 8.4% | 8.0% |

The body weight on the 17th day after the start of treatment was shown in Table 17.

TABLE 17
Body weight on the 17th day

| | | Weight (g)/Mean ± SEM | | |
|---|---|---|---|---|
| Group | Treatment | Day 0 ^{a} | Day 17 ^{a} | Changes |
| G1 | hIgG4 (2 mg/kg) | 24.9 ± 0.4 | 29.0 ± 0.6 | +4.1 |
| G2 | Antibody 2E5 (1 mg/kg) | 24.9 ± 0.7 | 28.6 ± 0.7 | +3.7 |
| G3 | Antibody 2E5 (2 mg/kg) | 25.0 ± 0.6 | 27.8 ± 0.7 | +2.8 |
| G4 | Lenvatinib mesylate | 24.9 ± 0.8 | 28.0 ± 0.8 | +3.1 |
| | (1 mg/kg) | | | |
| G5 | Lenvatinib mesylate | 24.9 ± 0.6 | 26.7 ± 0.6 | +1.8 |
| | (1 mg/kg) + | | | |
| | Antibody 2E5 (1 mg/kg) | | | |
| G6 | Lenvatinib mesylate | 25.0 ± 0.6 | 26.7 ± 0.7 | +1.7 |
| | (1 mg/kg) + | | | |
| | Antibody 2E5 (2 mg/kg) | | | |

Note:
^{a} Day 0 is the day of the start of treatment (i.e., the day of animal grouping). Day 17 is the 17th day after the start of treatment.

8.2. Tumor Volume Results

Tumor growth of all animals was closely monitored during the whole period of the experiment. No. 55191 mouse in the G1 group was euthanized in advance since the tumor volume exceeded 3000 mm³ on the 16th day after the start of treatment. The weight data of this mouse on the day of euthanasia was followed to the termination of the experiment and included in the statistical analysis. The tumor weight changes of all animals were shown in Table 18.

TABLE 18
Tumor volume

| | Animal | Days after the start of treatment/tumor volume (mm³) | | | | | | |
|---|---|---|---|---|---|---|---|---|
| Group | No. | 0 | 4 | 8 | 10 | 12 | 15 | 17 |
| G1 | 55106 | 76.5 | 224.2 | 891.7 | 1227.7 | 1601.3 | 1749.5 | 1706.5 |
| | 55115 | 76.1 | 208.8 | 489.8 | 817.4 | 956.0 | 2314.2 | 2792.2 |
| | 55136 | 70.8 | 221.7 | 690.8 | 998.5 | 1512.8 | 1919.3 | 2520.8 |
| | 55151 | 73.3 | 203.5 | 676.1 | 1085.3 | 1574.2 | 2049.0 | 3017.9 |
| | 55156 | 87.4 | 287.7 | 1037.1 | 1061.1 | 1360.2 | 1657.0 | 1820.8 |
| | 55157 | 79.3 | 289.7 | 1010.8 | 1027.8 | 1435.5 | 1912.4 | 3026.8 |
| | 55164 | 78.3 | 272.3 | 1139.6 | 1423.1 | 2509.8 | 2814.6 | 3998.8 |
| | 55188 | 80.5 | 223.6 | 1154.3 | 1683.4 | 1901.4 | 2538.0 | 3484.4 |
| | 55191 | 80.1 | 376.9 | 1297.1 | 1453.9 | 2725.9 | 3185.3 | 3185.3 |
| | 55194 | 69.4 | 133.6 | 350.5 | 345.5 | 574.2 | 681.8 | 838.1 |
| | Mean | 77 | 244 | 874 | 1112 | 1615 | 2082 | 2639 |
| | SD | 5 | 66 | 311 | 373 | 645 | 693 | 941 |
| | SEM | 2 | 21 | 98 | 118 | 204 | 219 | 298 |
| | CV | 6.8% | 26.9% | 35.5% | 33.5% | 39.9% | 33.3% | 35.7% |
| G2 | 55122 | 74.2 | 133.5 | 274.6 | 279.4 | 573.8 | 689.0 | 982.2 |
| | 55131 | 75.7 | 184.2 | 216.7 | 248.7 | 257.1 | 354.1 | 239.0 |
| | 55147 | 67.5 | 269.2 | 448.7 | 608.9 | 924.5 | 1149.7 | 1379.7 |
| | 55150 | 72.0 | 353.0 | 457.3 | 771.4 | 1312.1 | 1392.4 | 2172.7 |
| | 55161 | 77.2 | 183.0 | 233.1 | 234.7 | 381.6 | 591.7 | 565.9 |
| | 55168 | 80.6 | 140.7 | 511.8 | 717.8 | 1304.6 | 2234.1 | 2361.1 |
| | 55170 | 79.2 | 195.8 | 474.9 | 481.8 | 962.7 | 1105.7 | 1330.5 |
| | 55178 | 78.5 | 162.2 | 390.1 | 387.9 | 405.2 | 358.4 | 245.2 |
| | 55184 | 84.6 | 246.0 | 709.7 | 888.0 | 927.0 | 1634.1 | 2060.5 |
| | 55190 | 81.5 | 145.7 | 226.8 | 294.3 | 348.8 | 572.4 | 610.6 |
| | Mean | 77 | 201 | 394 | 491 | 740 | 1008 | 1195 |
| | SD | 5 | 69 | 159 | 240 | 397 | 613 | 797 |
| | SEM | 2 | 22 | 50 | 76 | 126 | 194 | 252 |
| | CV | 6.5% | 34.4% | 40.2% | 48.9% | 53.7% | 60.9% | 66.7% |
| | TGI | | 25.6% | 60.2% | 60.0% | 56.9% | 53.6% | 56.4% |
| G3 | 55108 | 70.6 | 147.7 | 226.8 | 288.3 | 653.9 | 741.2 | 1534.9 |
| | 55125 | 86.2 | 223.8 | 409.0 | 408.0 | 510.6 | 725.4 | 1149.8 |
| | 55135 | 72.3 | 413.3 | 613.1 | 836.3 | 1169.7 | 1205.5 | 1579.3 |
| | 55146 | 75.5 | 162.8 | 283.2 | 309.2 | 372.2 | 480.5 | 755.1 |
| | 55149 | 82.1 | 149.4 | 326.5 | 426.0 | 430.6 | 430.9 | 541.9 |
| | 55167 | 79.1 | 194.5 | 358.3 | 398.3 | 625.7 | 897.0 | 1359.9 |
| | 55171 | 76.6 | 263.1 | 948.9 | 1029.5 | 1663.8 | 2226.4 | 2487.6 |
| | 55173 | 80.1 | 199.2 | 430.6 | 526.7 | 1230.2 | 1474.0 | 1726.9 |
| | 55182 | 70.5 | 216.9 | 206.3 | 262.3 | 560.3 | 463.8 | 318.9 |
| | 55186 | 78.2 | 272.8 | 481.6 | 608.9 | 1092.6 | 1137.9 | 1568.4 |
| | Mean | 77 | 224 | 428 | 509 | 831 | 978 | 1302 |
| | SD | 5 | 79 | 220 | 251 | 429 | 560 | 638 |
| | SEM | 2 | 25 | 69 | 79 | 136 | 177 | 202 |
| | CV | 6.6% | 35.3% | 51.3% | 49.3% | 51.6% | 57.2% | 49.0% |
| | TGI | — | 11.9% | 55.9% | 58.2% | 51.0% | 55.1% | 52.2% |
| G4 | 55103 | 76.4 | 200.5 | 372.9 | 334.9 | 510.8 | 658.2 | 571.3 |
| | 55104 | 81.2 | 215.4 | 423.5 | 520.1 | 748.6 | 1225.3 | 1076.5 |
| | 55123 | 75.7 | 185.2 | 420.4 | 309.3 | 291.0 | 266.3 | 220.1 |
| | 55139 | 70.8 | 205.0 | 514.5 | 514.5 | 750.2 | 870.2 | 1285.2 |
| | 55155 | 75.3 | 220.4 | 383.5 | 484.3 | 820.6 | 887.5 | 1080.5 |
| | 55163 | 78.9 | 278.3 | 295.3 | 419.7 | 685.6 | 1155.8 | 1671.7 |
| | 55165 | 79.6 | 181.0 | 332.3 | 451.7 | 606.8 | 733.1 | 792.0 |
| | 55175 | 67.5 | 186.3 | 373.0 | 472.9 | 247.7 | 273.7 | 344.4 |
| | 55187 | 78.8 | 312.5 | 484.6 | 500.3 | 475.1 | 565.7 | 556.7 |
| | 55198 | 86.8 | 267.4 | 470.9 | 516.8 | 706.4 | 1040.9 | 1312.4 |
| | Mean | 77 | 225 | 407 | 452 | 584 | 768 | 891 |
| | SD | 5 | 45 | 69 | 76 | 198 | 335 | 470 |
| | SEM | 2 | 14 | 22 | 24 | 63 | 106 | 149 |
| | CV | 7.0% | 20.1% | 17.0% | 16.7% | 33.9% | 43.6% | 52.7% |
| | TGI | — | 11.3% | 58.6% | 63.7% | 67.0% | 65.6% | 68.2% |
| G5 | 55105 | 82.0 | 238.5 | 145.1 | 128.4 | 193.2 | 198.9 | 148.0 |
| | 55112 | 82.6 | 200.4 | 354.8 | 363.8 | 658.8 | 595.2 | 696.3 |
| | 55120 | 82.8 | 205.9 | 242.3 | 160.9 | 165.2 | 149.9 | 246.5 |
| | 55140 | 79.6 | 178.6 | 224.7 | 145.0 | 239.9 | 198.9 | 159.9 |
| | 55143 | 75.1 | 177.8 | 250.6 | 319.7 | 398.3 | 406.5 | 465.4 |
| | 55154 | 69.7 | 185.6 | 311.0 | 311.1 | 596.9 | 670.9 | 872.3 |
| | 55162 | 72.6 | 212.4 | 296.0 | 269.2 | 491.7 | 558.3 | 637.6 |
| | 55180 | 77.1 | 216.2 | 252.8 | 202.6 | 328.3 | 312.1 | 308.1 |
| | 55192 | 79.2 | 184.9 | 307.9 | 128.2 | 257.5 | 328.4 | 386.0 |
| | 55193 | 71.1 | 95.8 | 95.3 | 141.8 | 78.5 | 158.2 | 215.3 |
| | Mean | 77 | 190 | 248 | 217 | 341 | 358 | 414 |
| | SD | 5 | 38 | 79 | 90 | 192 | 193 | 249 |
| | SEM | 2 | 12 | 25 | 29 | 61 | 61 | 79 |
| | CV | 6.3% | 20.1% | 31.7% | 41.7% | 56.3% | 53.8% | 60.2% |
| | TGI | | 32.7% | 78.5% | 86.5% | 82.9% | 86.0% | 86.9% |
| G6 | 55101 | 81.2 | 356.9 | 265.5 | 205.2 | 179.2 | 225.1 | 189.4 |
| | 55109 | 76.4 | 244.8 | 209.1 | 199.1 | 277.8 | 292.9 | 368.1 |
| | 55113 | 78.8 | 151.6 | 171.2 | 205.8 | 237.7 | 242.8 | 290.4 |
| | 55121 | 76.2 | 224.3 | 148.5 | 288.4 | 342.6 | 294.7 | 236.5 |
| | 55127 | 72.2 | 251.4 | 310.9 | 178.7 | 346.6 | 470.5 | 379.5 |
| | 55159 | 69.4 | 144.5 | 208.0 | 147.0 | 237.0 | 276.8 | 230.2 |
| | 55160 | 79.8 | 246.6 | 363.8 | 313.5 | 850.6 | 1362.8 | 1484.6 |
| | 55185 | 73.6 | 139.9 | 127.3 | 155.7 | 215.3 | 271.0 | 197.3 |
| | 55189 | 85.6 | 97.7 | 125.5 | 134.5 | 181.4 | 159.9 | 168.8 |
| | 55199 | 78.5 | 202.5 | 256.4 | 226.6 | 391.0 | 446.8 | 624.8 |
| | Mean | 77 | 206 | 219 | 205 | 326 | 404 | 417 |
| | SD | 5 | 75 | 80 | 58 | 198 | 350 | 399 |
| | SEM | 1 | 24 | 25 | 18 | 63 | 111 | 126 |
| | CV | 6.1% | 36.6% | 36.6% | 28.4% | 60.8% | 86.5% | 95.7% |
| | TGI | — | 22.9% | 82.2% | 87.6% | 83.8% | 83.7% | 86.7% |

The tumor volume on the 17th day after the start of treatment was statistically analyzed using independent-samples t-test, and the result was shown in Table 19. There was a significant difference in the tumor volume between each treatment group and control group (P<0.05). There was a significant difference in the tumor volume between the combination group and the monotherapy group (P<0.05). There was no significant difference in the tumor volume between G2 and G3 (P>0.05).

TABLE 19
Synergy analysis

| | | Tumor volume | | | | |
|---|---|---|---|---|---|---|
| | | (mm³)/Mean ± SEM | | | | Synergy score |
| Group | Treatment | Day 0 ^{a} | Day 17 ^{a} | TGI_{TV} (%) | P^{b} | @ D17 |
| G1 | hIgG4 (2 mg/kg) | 77 ± 2 | 2639 ± 298 | — | — | |
| G2 | Antibody 2E5 (1 mg/kg) | 77 ± 2 | 1195 ± 252 | 56.4 | 0.002 | |
| G3 | Antibody 2E5 (2 mg/kg) | 77 ± 2 | 1302 ± 202 | 52.2 | 0.002 | |
| G4 | Lenvatinib mesylate (1 mg/kg) | 11 ± 2 | 891 ± 149 | 68.2 | <0.001 | |
| G5 | Lenvatinib mesylate (1 mg/kg) + | 77 ± 2 | 414 ± 79 | 86.9 | <0.001 | 1.18 |
| | antibody 2E5 (1 mg/kg) | | | | | |
| G6 | Lenvatinib mesylate (1 mg/kg) + | 77 ± 1 | 417 ± 126 | 86.7 | <0.001 | 1.15 |
| | Antibody 2E5 (2 mg/kg) | | | | | |

Note:
^{a} Day 0 is the day of the start of treatment, Day 17 is the 17th day after the start of treatment;
^{b}p value vs control group (G1);
Difference between groups: G2 versus G3, p = 0.743; G5 versus G6, p = 0.982; G2 versus G5, p = 0.013; G4 versus G5, p = 0.013; G3 versus G6, p = 0.002; G4 versus G6, p = 0.026.

Synergistic effect between Lenvatinib mesylate and antibody 2E5 was demonstrated as synergy score was larger than 1 in the combination groups G5 and G6.

8.3. Tumor Weight Results

In this experiment, all animals were euthanized on the 17th day after the start of treatment, and tumors were separated, weighed and photographed. The individual tumor weight data was shown in Table 20.

TABLE 20
Tumor weight

| Group | Animal No. | Tumor weight (g) |
|---|---|---|
| G1 | 55106 | 1.228 |
| | 55115 | 2.179 |
| | 55136 | 1.873 |
| | 55151 | 2.523 |
| | 55156 | 1.199 |
| | 55157 | 2.062 |
| | 55164 | 3.519 |
| | 55188 | 2.413 |
| | 55191 | 3.376 |
| | 55194 | 0.485 |
| | Mean | 2.086 |
| | SD | 0.952 |
| | SEM | 0.301 |
| | CV | 0.456 |
| G2 | 55122 | 0.949 |
| | 55131 | 0.097 |
| | 55147 | 1.273 |
| | 55150 | 1.466 |
| | 55161 | 0.419 |
| | 55168 | 1.747 |
| | 55170 | 0.914 |
| | 55178 | 0.275 |
| | 55184 | 1.549 |
| | 55190 | 0.397 |
| | Mean | 0.909 |
| | SD | 0.589 |
| | SEM | 0.186 |
| | CV | 0.648 |
| | TGI_{TW} | 56.4% |
| G3 | 55108 | 0.970 |
| | 55125 | 0.809 |
| | 55135 | 1.323 |
| | 55146 | 0.579 |
| | 55149 | 0.480 |
| | 55167 | 0.969 |
| | 55171 | 2.501 |
| | 55173 | 1.475 |
| | 55182 | 0.520 |
| | 55186 | 1.175 |
| | Mean | 1.080 |
| | SD | 0.602 |
| | SEM | 0.190 |
| | CV | 0.558 |
| | TGI_{TW} | 48.2% |
| G4 | 55103 | 0.685 |
| | 55104 | 0.913 |
| | 55123 | 0.259 |
| | 55139 | 0.862 |
| | 55155 | 0.739 |
| | 55163 | 1.368 |
| | 55165 | 0.745 |
| | 55175 | 0.499 |
| | 55187 | 0.465 |
| | 55198 | 1.137 |
| | Mean | 0.767 |
| | SD | 0.326 |
| | SEM | 0.103 |
| | CV | 0.425 |
| | TGI_{TW} | 63.2% |
| G5 | 55105 | 0.133 |
| | 55112 | 0.762 |
| | 55120 | 0.172 |
| | 55140 | 0.177 |
| | 55143 | 0.634 |
| | 55154 | 0.851 |
| | 55162 | 0.669 |
| | 55180 | 0.508 |
| | 55192 | 0.563 |
| | 55193 | 0.162 |
| | Mean | 0.463 |
| | SD | 0.277 |
| | SEM | 0.088 |
| | CV | 0.598 |
| | TGI_{TW} | 77.8% |
| G6 | 55101 | 0.164 |
| | 55109 | 0.345 |
| | 55113 | 0.250 |
| | 55121 | 0.226 |
| | 55127 | 0.409 |
| | 55159 | 0.325 |
| | 55160 | 0.993 |
| | 55185 | 0.194 |
| | 55189 | 0.381 |
| | 55199 | 0.456 |
| | Meam | 0.374 |
| | SD | 0.238 |
| | SEM | 0.075 |
| | CV | 0.635 |
| | TGI_{TW} | 82.1% \| |

The tumor weight was statistically analyzed using independent-samples t-test, and the result was shown in Table 21. There was a significant difference in the tumor weight between the combination group and monotherapy group (p<0.05).

TABLE 21
tumor weight analysis

| | | Tumor weight (g) | | |
|---|---|---|---|---|
| Group | Treatment | Mean ± SEM | TGI_{TV} (%) | P^{a} |
| G1 | IgG4 (2 mg/kg) | 2.086 ± 0.301 | — | — |
| G2 | Antibody 2E5 (1 mg/kg) | 0.909 ± 0.186 | 56.4 | 0.004 |
| G3 | Antibody 2E5 (2 mg/kg) | 1.080 ± 0.190 | 48.2 | 0.011 |
| G4 | Lenvatinib mesylate | 0.767 ± 0.103 | 63.2 | 0.002 |
| | (1 mg/kg) | | | |
| G5 | Lenvatinib mesylate | 0.463 ± 0.088 | 77.8 | <0.001 |
| | (1 mg/kg) + | | | |
| | Antibody 2E5 (1 mg/kg) | | | |
| G6 | Lenvatinib mesylate | 0.374 ± 0.075 | 82.1 | <0.001 |
| | (1 mg/kg) + | | | |
| | Antibody 2E5 (2 mg/kg) | | | |

Note:
^{a}p value vs control group (G1);
Difference between groups: G2 versus G3, p = 0.528;
G5 versus G6, p = 0.451;
G2 versus G5, p = 0.0499;
G4 versus G5, p = 0.037;
G3 versus G6, p = 0.003;
G4 versus G6, p = 0.006.

It is to be understood that the foregoing description of the preferred embodiments is intended to be purely illustrative of the principles of the disclosure, rather than exhaustive thereof, and that changes and variations will be apparent to those skilled in the art, and that the present disclosure is not intended to be limited other than expressly set forth in the following claims.

1. A pharmaceutical combination comprising a substance A and a substance B;
wherein the substance A is Compound F, a crystal form thereof, a pharmaceutically acceptable salt thereof or a solvate thereof; 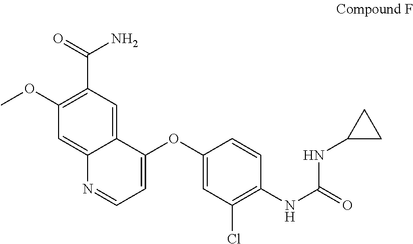 [SMILES: COc1cc2nccc(Oc3ccc(NC(=O)NC4CC4)c(Cl)c3)c2cc1C(N)=O]
the substance B is an antibody M comprising a heavy chain CDR amino acid sequence selected from the group consisting of: SEQ ID NOs: 1, 3, 5, 7, 9, 11, 13, 15, 17, 19, 21 and 23, or an antigen binding fragment thereof.

2. The pharmaceutical combination as defined in claim 1, wherein the antibody M comprises:
(a) a heavy chain variable region comprising a HCDR1 amino acid sequence of SEQ ID NO: 1, a HCDR2 amino acid sequence of SEQ ID NO: 3, and a HCDR3 amino acid sequence of SEQ ID NO: 5; and a light chain variable region comprising a LCDR1 amino acid sequence of SEQ ID NO: 2, a LCDR2 amino acid sequence of SEQ ID NO: 4, and a LCDR3 amino acid sequence of SEQ ID NO: 6;
(b) a heavy chain variable region comprising a HCDR1 amino acid sequence of SEQ ID NO: 7, a HCDR2 amino acid sequence of SEQ ID NO: 9, and a HCDR3 amino acid sequence of SEQ ID NO: 11; and a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12;
(c) a heavy chain variable region comprising a HCDR1 amino acid sequence of SEQ ID NO: 13, a HCDR2 amino acid sequence of SEQ ID NO: 15, and a HCDR3 amino acid sequence of SEQ ID NO: 17; and a light chain variable region comprising a LCDR1 amino acid sequence of SEQ ID NO: 14, a LCDR2 amino acid sequence of SEQ ID NO: 16, and a LCDR3 amino acid sequence of SEQ ID NO: 18; or
(d) a heavy chain variable region comprising a HCDR1 amino acid sequence of SEQ ID NO: 19, a HCDR2 amino acid sequence of SEQ ID NO: 21, and a HCDR3 amino acid sequence of SEQ ID NO: 23; and a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12;
optionally, the antibody M comprises a heavy chain variable region comprising a HCDR1 amino acid sequence of SEQ ID NO: 7, a HCDR2 amino acid sequence of SEQ ID NO: 9, and a HCDR3 amino acid sequence of SEQ ID NO: 11; and a light chain variable region comprising a LCDR1 amino acid sequence of SEQ ID NO: 8, a LCDR2 amino acid sequence of SEQ ID NO: 10, and a LCDR3 amino acid sequence of SEQ ID NO: 12.

3. The pharmaceutical combination as defined in claim 1 or 2, wherein the antibody M comprises:
(a) a heavy chain variable region comprising an amino acid sequence of SEQ ID NO: 20; and a light chain variable region comprising an amino acid sequence of SEQ ID NO: 22;
(b) a heavy chain variable region comprising an amino acid sequence of SEQ ID NO: 24; and a light chain variable region comprising an amino acid sequence of SEQ ID NO: 25;
(c) a heavy chain variable region comprising an amino acid sequence of SEQ ID NO: 26; and a light chain variable region comprising an amino acid sequence of SEQ ID NO: 27; or
(d) a heavy chain variable region comprising an amino acid sequence of SEQ ID NO: 28; and a light chain variable region comprising an amino acid sequence of SEQ ID NO: 25;
optionally, the antibody M comprises a heavy chain variable region comprising an amino acid sequence of SEQ ID NO: 24; and a light chain variable region comprising an amino acid sequence of SEQ ID NO: 25.

4. The pharmaceutical combination as defined in any one of claims 1-3, wherein the antibody M comprises a heavy chain constant region of human IgG4, and a light chain constant region of human λ or κ light chain;
and/or, the substance B is the antibody M;
and/or, the substance A is the mesylate of Compound F;
and/or, the pharmaceutical combination further comprises a pharmaceutical excipient.

5. The pharmaceutical combination as defined in any one of claims 1-4, wherein the pharmaceutical combination is in the form of a fixed combination or a non-fixed combination, optionally, in the form of a non-fixed combination.

6. A pharmaceutical composition comprising a substance A and a substance B; wherein the substance A and the substance B are as defined in any one of claims 1-4.

7. A kit comprising:
a first container comprising a first pharmaceutical composition comprising a substance A; and
a second container comprising a second pharmaceutical composition comprising a substance B;
wherein the substance A and the substance B are as defined in any one of claims 1-4.

8. A method for treating a cancer comprising administering the pharmaceutical combination as defined in any one of claims 1-5 or the pharmaceutical composition as defined in claim 6.

9. The method as defined in claim 8, wherein the cancer is non-small cell lung cancer, small cell lung cancer, renal cell cancer, colorectal cancer, colon cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas or myelomas; optionally, the cancer is colon cancer;
and/or, the substance A and the substance B are administrated simultaneously or separately, optionally, administrated separately;
and/or, the substance A is administrated orally;
and/or, the substance B is administrated by injection.

10. A pharmaceutical combination comprising a substance A and a substance C;
wherein the substance A is Compound F, a crystal form thereof, a pharmaceutically acceptable salt thereof or a solvate thereof; 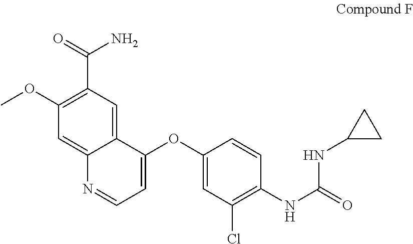 [SMILES: COc1cc2nccc(Oc3ccc(NC(=O)NC4CC4)c(Cl)c3)c2cc1C(N)=O]
the substance C is an antibody N comprising a CDR amino acid sequence selected from the group consisting of SEQ ID NOs: 38, 39, 40, 41, 42, 43, 44, 45, 46, 47, 48, 49, 50 and 51, or an antigen binding fragment thereof.

11. The pharmaceutical combination as defined in claim 10, wherein the antibody N comprises:
(a) a heavy chain variable region comprising a HCDR1 amino acid sequence of SEQ ID NO: 38, a HCDR2 amino acid sequence of SEQ ID NO: 39, and a HCDR3 amino acid sequence selected from the group consisting of SEQ ID NOs: 40 and 41; and
(b) a light chain variable region comprising a LCDR1 amino acid sequence selected from the group consisting of SEQ ID NOs: 42, 43, 44, 45 and 46, a LCDR2 amino acid sequence of SEQ ID NO: 47, and a LCDR3 amino acid sequence selected from the group consisting of SEQ ID NOs: 48, 49, 50 and 51.

12. The pharmaceutical combination as defined in claim 10 or 11, wherein the antibody N comprises:
(a) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence of SEQ ID NO: 42, a LCDR2 amino acid sequence of SEQ ID NO: 47, and a LCDR3 amino acid sequence of SEQ ID NO: 48;
(b) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 42, a LCDR2 amino acid sequence of SEQ ID NO: 47, and a LCDR3 amino acid sequence of SEQ ID NO: 49;
(c) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 43, a LCDR2 amino acid sequence of SEQ ID NO: 47, and a LCDR3 amino acid sequence of SEQ ID NO: 49;
(d) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 44, a LCDR2 amino acid sequence of SEQ ID NO: 47, and a LCDR3 amino acid sequence of SEQ ID NO: 49;
(e) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 49;
(f) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 44, a LCDR2 amino acid sequence of SEQ ID NO: 47, and a LCDR3 amino acid sequence of SEQ ID NO: 49;
(g) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 49;
(h) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 45, a LCDR2 amino acid sequence of SEQ ID NO: 47, and a LCDR3 amino acid sequence of SEQ ID NO: 50;
(i) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 46, a LCDR2 amino acid sequence of SEQ ID NO: 47, and a LCDR3 amino acid sequence of SEQ ID NO: 51; or
(j) a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 40; and a light chain variable region comprising a LCDR1 amino acid sequence selected from the group consisting of SEQ ID NO: 46, a LCDR2 amino acid sequence of SEQ ID NO: 47, and a LCDR3 amino acid sequence of SEQ ID NO: 48;
optionally, the antibody N comprises a heavy chain variable region comprising a HCDR1 amino acid sequence of SEQ ID NO: 38, a HCDR2 amino acid sequence of SEQ ID NO: 39, and a HCDR3 amino acid sequence of SEQ ID NO: 41; and a light chain variable region comprising a LCDR1 amino acid sequence selected from the group consisting of SEQ ID NO: 42, a LCDR2 amino acid sequence of SEQ ID NO: 47, and a LCDR3 amino acid sequence of SEQ ID NO: 49.

13. The pharmaceutical combination as defined in any one of claims 10-12, wherein the antibody N comprises:
(a) a heavy chain variable region comprising an amino acid sequence of SEQ ID NO: 29; and a light chain variable region comprising an amino acid sequence of SEQ ID NO: 31;
(b) a heavy chain variable region comprising an amino acid sequence of SEQ ID NO: 30; and a light chain variable region comprising an amino acid sequence of SEQ ID NO: 31;
(c) a heavy chain variable region comprising an amino acid sequence of SEQ ID NO: 30; and a light chain variable region comprising an amino acid sequence of SEQ ID NO: 32;
(d) a heavy chain variable region comprising an amino acid sequence of SEQ ID NO: 30; and a light chain variable region comprising an amino acid sequence of SEQ ID NO: 33;
(e) a heavy chain variable region comprising an amino acid sequence of SEQ ID NO: 29; and a light chain variable region comprising an amino acid sequence of SEQ ID NO: 34;
(f) a heavy chain variable region comprising an amino acid sequence of SEQ ID NO: 29; and a light chain variable region comprising an amino acid sequence of SEQ ID NO: 33;
(g) a heavy chain variable region comprising an amino acid sequence of SEQ ID NO: 30; and a light chain variable region comprising an amino acid sequence of SEQ ID NO: 34;
(h) a heavy chain variable region comprising an amino acid sequence of SEQ ID NO: 30; and a light chain variable region comprising an amino acid sequence of SEQ ID NO: 35;
(i) a heavy chain variable region comprising an amino acid sequence of SEQ ID NO: 29; and a light chain variable region comprising an amino acid sequence of SEQ ID NO: 36; or
(j) a heavy chain variable region comprising an amino acid sequence of SEQ ID NO: 30; and a light chain variable region comprising an amino acid sequence of SEQ ID NO: 37;
optionally, the antibody N comprises a heavy chain variable region comprising an amino acid sequence of SEQ ID NO: 30; and a light chain variable region comprising an amino acid sequence of SEQ ID NO: 31.

14. The pharmaceutical combination as defined in any one of claims 10-13, wherein the antibody N further comprises a heavy chain constant region of human IgG4, and a light chain constant region of human λ or κ light chain;
and/or, the substance C is the antibody N;
and/or, the substance A is the mesylate of Compound F;
and/or, the pharmaceutical combination further comprises a pharmaceutical excipient.

15. The pharmaceutical combination as defined in any one of claims 10-14, wherein the pharmaceutical combination is in the form of a fixed combination or a non-fixed combination, optionally, in the form of a non-fixed combination.

16. A pharmaceutical composition comprising a substance A and a substance C; wherein the substance A and the substance C are as defined in any one of claims 10-14.

17. A kit comprising:
a first container comprising a first pharmaceutical composition comprising a substance A; and
a second container comprising a second pharmaceutical composition comprising a substance C;
wherein the substance A and the substance C are as defined in any one of claims 10-14.

18. A method for treating a cancer comprising administering the pharmaceutical combination as defined in any one of claims 10-15 or the pharmaceutical composition as defined in claim 16.

19. The method as defined in claim 18, wherein the cancer is non-small cell lung cancer, small cell lung cancer, renal cell cancer, colorectal cancer, colon cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas or myelomas; optionally, the cancer is colon cancer;
and/or, the substance A and the substance C are administrated simultaneously or separately, optionally, administrated separately;
and/or, the substance A is administrated orally;
and/or, the substance C is administrated by injection.


SEQUENCE LISTING (51 sequences)

SEQ ID NO: 1 - protein, 7 aa, Homo sapiens
IRTYYWG

SEQ ID NO: 2 - protein, 11 aa, Homo sapiens
SGDKLGDKYAC

SEQ ID NO: 3 - protein, 16 aa, Homo sapiens
YIYYSGSTRYNPSLKS

SEQ ID NO: 4 - protein, 7 aa, Homo sapiens
QDTKRPS

SEQ ID NO: 5 - protein, 7 aa, Homo sapiens
LSYFFDY

SEQ ID NO: 6 - protein, 9 aa, Homo sapiens
QAWDSGTVI

SEQ ID NO: 7 - protein, 5 aa, Homo sapiens
SYAMS

SEQ ID NO: 8 - protein, 11 aa, Homo sapiens
GGNNIGSKSVH

SEQ ID NO: 9 - protein, 17 aa, Homo sapiens
GISGSGGFTYYADSVKG

SEQ ID NO: 10 - protein, 7 aa, Homo sapiens
DDSDRPS

SEQ ID NO: 11 - protein, 12 aa, Homo sapiens
PPRGYNYGPFDY

SEQ ID NO: 12 - protein, 11 aa, Homo sapiens
QVWDSSSDHVV

SEQ ID NO: 13 - protein, 7 aa, Homo sapiens
SISNYWG

SEQ ID NO: 14 - protein, 11 aa, Homo sapiens
SGDKLGDKYAC

SEQ ID NO: 15 - protein, 16 aa, Homo sapiens
SIYYSGSTNYNPPLKS

SEQ ID NO: 16 - protein, 7 aa, Homo sapiens
QDSKRPS

SEQ ID NO: 17 - protein, 7 aa, Homo sapiens
LTYYFDY

SEQ ID NO: 18 - protein, 9 aa, Homo sapiens
QTWDSSTVV

SEQ ID NO: 19 - protein, 5 aa, Homo sapiens
SYAMS

SEQ ID NO: 20 - protein, 117 aa, Homo sapiens
QLQLQESGPGLVKPSESLSLTCTVSGGSISIRTYYWGWIRQPPGTGLEWMGYIYYSGSTRYNPSLKSRVTISVDTSKNQFSLKLSSVTAADTAVYYCARLSYFFDYWGQGTLVTVSS

SEQ ID NO: 21 - protein, 17 aa, Homo sapiens
GFSGSGFITYYADSVKG

SEQ ID NO: 22 - protein, 106 aa, Homo sapiens
SYELTQPPSVSVSPGQTASITCSGDKLGDKYACWYQQKPGQSPVMVIYQDTKRPSGIPERFSGSNSGNTATLTISGTLAMDEADYYCQAWDSGTVIFGGGTKLTVL

SEQ ID NO: 23 - protein, 12 aa, Homo sapiens
PPRGYNYGPFDY

SEQ ID NO: 24 - protein, 121 aa, Homo sapiens
EVQLLESGGGLVQPGGSLRLSCAASGFTFSSYAMSWVRQAPGKGLEWVSGISGSGGFTYYADSVKGRFTISRDNSKNTLYLQMNSLRAEDTAVYYCAKPPRGYNYGPFDYWGQGTLVTVSS

SEQ ID NO: 25 - protein, 108 aa, Homo sapiens
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 26 - protein, 117 aa, Homo sapiens
QLQLQESGPGLVKPSETLSLTCTVSGGSISSISNYWGWIRQPPGKGLEWIGSIYYSGSTNYNPPLKSRVTISVDTTKNQFSLKLSSVTAADTAVYYCARLTYYFDYWGQGMLVTVSS

SEQ ID NO: 27 - protein, 106 aa, Homo sapiens
SYDLTQPPSVSVSPGQTASITCSGDKLGDKYACWYQQKPGQSPLLVIQQDSKRPSGIPARFSGSNSGNTATLTISGTQAMDEADYFCQTWDSSTVVFGGGTKLTVL

SEQ ID NO: 28 - protein, 121 aa, Homo sapiens
EVQLLESGGGLVQPGGSLRLSCAASGFTFSSYAMSWVRQAPGKGLEWVSGFSGSGFITYYADSVKGRFTISRDNSKNTLYLQMNSLRAEDTAVYYCAMPPRGYNYGPFDYWGQGTLVTVSS

SEQ ID NO: 29 - protein, 115 aa, Artificial Sequence
  heavy chain variable region
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAILGYFDYWGQGTMVTVSS

SEQ ID NO: 30 - protein, 115 aa, Artificial Sequence
  heavy chain variable region
QVQLVQSGAEVKKPGSSVKVSCKASGFTFTTYYISWVRQAPGQGLEYLGYINMGSGGTNYNEKFKGRVTITADKSTSTAYMELSSLRSEDTAVYYCAIIGYFDYWGQGTMVTVSS

SEQ ID NO: 31 - protein, 112 aa, Artificial Sequence
  light chain variable region
DVVMTQSPLSLPVTLGQPASISCRSSQSLLDSDGGTYLYWFQQRPGQSPRRLIYLVSTLGSGVPDRFSGSGSGTDFTLKISRVEAEDVGVYYCMQLTHWPYTFGQGTKLEIK

SEQ ID NO: 32 - protein, 112 aa, Artificial Sequence
  light chain variable region
DVVMTQSPLSLPVTLGQPASISCRSSQSLLDSDGSTYLYWFQQRPGQSPRRLIYLVSTLGSGVPDRFSGSGSGTDFTLKISRVEAEDVGVYYCMQLTHWPYTFGQGTKLEIK

SEQ ID NO: 33 - protein, 112 aa, Artificial Sequence
  light chain variable region
DVVMTQSPLSLPVTLGQPASISCRSSQSLLDSDGATYLYWFQQRPGQSPRRLIYLVSTLGSGVPDRFSGSGSGTDFTLKISRVEAEDVGVYYCMQLTHWPYTFGQGTKLEIK

SEQ ID NO: 34 - protein, 112 aa, Artificial Sequence
  light chain variable region
DVVMTQSPLSLPVTLGQPASISCRSSQSLLDSDGNTYLYWFQQRPGQSPRRLIYLVSTLGSGVPDRFSGSGSGTDFTLKISRVEAEDVGVYYCMQLTHWPYTFGQGTKLEIK

SEQ ID NO: 35 - protein, 112 aa, Artificial Sequence
  light chain variable region
DVVMTQSPLSLPVTLGQPASISCRSSQSLLDSDGNTYLYWFQQRPGQSPRRLIYLVSTLGSGVPDRFSGSGSGTDFTLKISRVEAEDVGVYYCMQLTHAPYTFGQGTKLEIK

SEQ ID NO: 36 - protein, 112 aa, Artificial Sequence
  light chain variable region
DVVMTQSPLSLPVTLGQPASISCRSSQSLLDSDGQTYLYWFQQRPGQSPRRLIYLVSTLGSGVPDRFSGSGSGTDFTLKISRVEAEDVGVYYCMQLTHEPYTFGQGTKLEIK

SEQ ID NO: 37 - protein, 112 aa, Artificial Sequence
  light chain variable region
DVVMTQSPLSLPVTLGQPASISCRSSQSLLDSDGQTYLYWFQQRPGQSPRRLIYLVSTLGSGVPDRFSGSGSGTDFTLKISRVEAEDVGVYYCMQLTHENYTFGQGTKLEIK

SEQ ID NO: 38 - protein, 5 aa, Artificial Sequence
  HCDR1
TYYIS

SEQ ID NO: 39 - protein, 17 aa, Artificial Sequence
  HCDR2
YINMGSGGTNYNEKFKG

SEQ ID NO: 40 - protein, 6 aa, Artificial Sequence
  HCDR3
LGYFDY

SEQ ID NO: 41 - protein, 6 aa, Artificial Sequence
  HCDR3
IGYFDY

SEQ ID NO: 42 - protein, 16 aa, Artificial Sequence
  LCDR1
RSSQSLLDSDGGTYLY

SEQ ID NO: 43 - protein, 16 aa, Artificial Sequence
  LCDR1
RSSQSLLDSDGSTYLY

SEQ ID NO: 44 - protein, 16 aa, Artificial Sequence
  LCDR1
RSSQSLLDSDGATYLY

SEQ ID NO: 45 - protein, 16 aa, Artificial Sequence
  LCDR1
RSSQSLLDSDGNTYLY

SEQ ID NO: 46 - protein, 16 aa, Artificial Sequence
  LCDR1
RSSQSLLDSDGQTYLY

SEQ ID NO: 47 - protein, 7 aa, Artificial Sequence
  LCDR2
LVSTLGS

SEQ ID NO: 48 - protein, 9 aa, Artificial Sequence
  LCDR3
MQLTHENYT

SEQ ID NO: 49 - protein, 9 aa, Artificial Sequence
  LCDR3
MQLTHWPYT

SEQ ID NO: 50 - protein, 9 aa, Artificial Sequence
  LCDR3
MQLTHAPYT

SEQ ID NO: 51 - protein, 9 aa, Artificial Sequence
  LCDR3
MQLTHEPYT